(19) United States
(12) Patent Application Publication
(10) Pub. No.: US 2015/0191793 A1
(43) Pub. Date: Jul. 9, 2015

(54) MOLECULAR MARKERS FOR PROGNOSTICALLY PREDICTING PROSTATE CANCER, METHOD AND KIT THEREOF

(71) Applicant: NATIONAL HEALTH RESEARCH INSTITUTES, Miaoli County (TW)

(72) Inventors: Kun-Chih Kelvin TSAI, Miaoli County (TW); Chi-Rong LI, Taipei (TW); Jiun-Ming Jimmy SU, Miaoli County (TW)

(21) Appl. No.: 14/568,075

(22) Filed: Dec. 11, 2014

Related U.S. Application Data
(62) Division of application No. 13/853,548, filed on Mar. 29, 2013, now abandoned.
(60) Provisional application No. 61/617,293, filed on Mar. 29, 2012.

(51) Int. Cl. C12Q 1/68; G01N 33/574

(52) U.S. Cl. CPC C12Q 1/6886; G01N 33/57434; C12Q 2600/158; C12Q 2600/118; G01N 2800/52

(57) ABSTRACT

The present application provides a method for predicting clinical prognosis for a human subject diagnosed with prostate cancer, comprising: detecting an expression level of a marker gene selected from a group consisting of ABCG1, PDCD4, KLF6, ST6, BTD, BANF1, IRS1, ZNF185, ANXA11, DUSP2, KLF4 and DSC2, in a biological sample containing prostate cancer cells obtained from the human subject; and predicting a likehood of the clinical prognosis by comparing the expression level of the marker gene with a reference level. The present application also provides a combination of molecular markers and a kit containing thereof.

### CROSS-REFERENCE TO RELATED APPLICATION(S)

This application is a divisional application of U.S. application Ser. No. 13/853,548, filed on Mar. 29, 2013, which application claims priority to U.S. Provisional Application No. 61/617,293 filed on Mar. 29, 2012, each of which is incorporated herein by reference in its entirety.

### BACKGROUND OF THE INVENTION

1. Field of the Invention

The present invention relates to novel molecular markers of prostate cancer, and a method and a kit for detection of prostate cancer comprising the molecular markers.

2. Description of the Related Art

Prostate cancer is a leading cause of cancer-related death in men. For early-stage, localized prostate cancer, radical prostatectomy offers an opportunity of eradicating the disease. However, approximately 15-30% of patients with initially localized diseases develop recurrence within 5-10 years, resulting in poor therapeutic outcomes (Bill-Axelson et al., 2005; Pound et al., 1999). Further improvements in the prognosis of patients with prostate cancer may rely on a deeper understanding of the patho-molecular mechanisms underlying disease recurrence as well as rationalized treatment plans based on a better prediction of the clinical behaviors of human prostate cancer.

Like most glandular cancers, the malignant transformation of prostatic epithelium involves a gradual and variable loss of the normal glandular architectures. As such human prostate cancer frequently displays considerable intra-tumoral heterogeneity in glandular differentiation, a factor widely used for the pathological classification of prostate cancer such as the Gleason grading system (Gleason, 1992). Large scale clinical studies have established the degree of glandular differentiation as a determinant of the clinical behaviors of prostate cancer. Specifically, poorly to differentiated, high-Gleason-grade tumors were associated with higher probabilities of tumor recurrence and poor prognosis (Albertsen et al., 1995; Stamey et al., 1999). This morphology-based classification system, however, is only modestly prognostic and does not allow for risk stratification of prostate cancer with similar histopathological characteristics. Assessments of tissue architectures did not provide functional or mechanistic insights into observed tumor variations. There is thus a critical need for pathway-informed and molecularly-based diagnostic assays with increased accuracy in the prediction of clinical outcome in prostate cancer.

Recently, high throughput genomic profiling techniques have facilitated the molecular characterization of human malignant tumors, including prostate cancer (Glinsky et al., 2004; Henshall et al., 2003; Singh et al., 2002; Stratford et al., 2010; van 't Veer et al., 2002; van de Vijver et al., 2002). The profound prognostic utilities of these genomic markers point to the intrinsic molecular characteristic of tumors as a crucial determinant to their clinical behaviors (Ramaswamy et al., 2003). For instance, by comparing gene expression profiles of prostate cancer specimen and normal adjacent prostate, Dhanasekaran et al. identified clusters of coordinately expressed genes of prostate cancer (Dhanasekaran et al., 2001). Two of these genes, including hepsin (HPN) and pim-1 (PIM1), were shown to correlate with measures of clinical outcome. Similarly, by comparing the gene expression patterns of metastatic prostate cancer and localized prostate cancer, Varambally et al. identified 55 upregulated genes and 480 downregulated genes (Varambally et al., 2002). Focusing on the top-ranked genes they experimentally verified enhancer of Zeste homolog 2 (EZH2) as a metastasis-promoting gene and a prognostic marker in prostate cancer. Studying gene expression patterns of tumors from 21 patients with prostate cancer who received radical prostatectomy, Singh et al. established a 5-gene model that predicted risk of post-operative disease recurrence with an accuracy reaching 90% (Singh et al., 2002). This model was established based on few tumor samples and its performance had not been verified in independent patient cohorts. Based upon the same set of 21 prostate cancer tumor samples, Glinsky et al. identified three sets of genes by comparing gene-expression profiles in tumors from patients with recurrent versus nonrecurrent prostate cancer (Glinsky et al., 2004). These gene signatures were able to discriminate human prostate cancers exhibiting recurrent or nonrecurrent clinical behaviors with 86-95% accuracy. Using a small number of tumor samples including four from patients with recurring prostate cancer and five from those with non-recurring tumors, Gary et al. identified a set of 33 genes that differentially expressed between the two groups of prostate cancer (US Patent Application US 2010/0196902 A1). This gene signature of prostate cancer also suffered from the small sample size and the lack of independent verification.

Aside from the development of molecular markers, genomic tools can also be used to molecularly define tumor subtypes or distinguish among primary and metastatic prostate cancers. For example, transcript profiling of human prostate cancer tissues has supported the existence of three distinct tumor subclasses that were associated with tumor grades and stages (Lapointe et al., 2004). LaTulippe et al. identified more than 3000 genes that were differentially expressed between primary and metastatic prostate cancers (LaTulippe et al., 2002). Gene expression patterns of tumor differentiation as reflected by the Gleason scores have also been described. For instance, gene expression profiling of 29 microdissected prostate tumors corresponding led to the identification of a 86-gene model capable of distinguishing low-grade from high-grade prostate cancer (True et al., 2006). It should be noted that the above mentioned molecular patterns were identified from clinical prostate tumor specimen and might only reflect established tumor characteristics without providing mechanisms underlying the pathogenesis of these tumor variations. In this regard, knowledge-based approaches offer an opportunity to identify more rational markers or classification systems that benefit clinical decision-making and therapeutic advancement. Such approaches have been used to establish the prognostic roles of gene profiles associated with tumor progenitor cells, stromal activation or tissue differentiation in several types of solid tumors (Chang et al., 2004; Fournier et al., 2006; Liu et al., 2007; Sotiriou et al., 2006).

Currently prevailing models of tumorigenesis suggest that tissue differentiation and tumor progression share similar gene regulations and molecular pathways. Molecular changes associated with the differentiation process of glandular epithelium may be difficult to study in vivo. However, a physiological relevant three-dimensional organotypic culture model has been used to recapitulate the structural and functional differentiation processes of mammary acini, the basic structural unit of normal mammary epithelium (Debnath and Brugge, 2005; Lee et al., 2007). Similar models have successfully recapitulated the morphogenetic and differentiation processes of prostate, pancreatic and pulmonary epithelium (Gutierrez-Barrera et al., 2007; Mondrinos et al., 2006; Webber et al., 1997). Comparative gene expression analysis using this developmental model has led to the identification of gene expression profiles and marker genes that showed significant association with breast cancer prognosis (Fournier et al., 2006; Kenny et al., 2007). Whether or not the same paradigm can be applied to other types of glandular cancers, such as prostate cancer, remains unclear.

Therefore, it still needs molecular markers for predicting the clinical outcomes of prostate cancer, such as recurrence, with improved accuracy and clinical applicability.

### SUMMARY

The present application describes a method for predicting clinical prognosis for a human subject diagnosed with prostate cancer, comprising: detecting an expression level of a marker gene selected from a group consisting of ABCG1, PDCD4, KLF6, ST6, BTD, BANF1, IRS1, ZNF185, ANXA11, DUSP2, KLF4 and DSC2, in a biological sample containing prostate cancer cells obtained from the human subject; and predicting a likelihood of the clinical prognosis by comparing the expression level of the marker gene with a reference level. The biological sample can be obtained by aspiration, biopsy, or surgical resection.

The present application also provides a combination of molecular markers for predicting clinical prognosis of prostate cancer, comprising at least two of marker genes ABCG1, PDCD4, KLF6, ST6, BTD, BANF1, IRS1, ZNF185, ANXA11, DUSP2, KLF4 and DSC2.

The present application further provides a kit for predicting clinical prognosis of prostate cancer, comprising a means for detecting an expression level of a marker gene selected from a group consisting of ABCG1, PDCD4, KLF6, ST6, BTD, BANF1, IRS1, ZNF185, ANXA11, DUSP2, KLF4 and DSC2.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIGS. 1A and 1B shows the structural organization of prostate epithelial cells using the three-dimensional culture model. FIG. 1A shows representative confocal images of RWPE-1 cell clusters (formed at 48 hours in culture) and acini (formed at day 6 in culture) in three-dimensional reconstituted basement membrane matrices (upper panels). The lower panels show confocal images of prostate cancer LNCaP cell clusters (formed at 48 hours in culture) or spheroids (formed at day 6 in culture) in three-dimensional reconstituted basement membrane matrices. The structures were immunostained with basal extracellular matrix receptor α6-integrin (red) and the apical marker GM130 (green). Nuclei were counterstained with Hoechst 33342 (blue). Scale bars, 20 μm. FIG. 1B shows percent polarized organoids formed by RWPE-1 cells or LNCaP cells as quantified by visual examination and counting under a fluorescence microscope. Data are represented as mean±SEM. n=3. ***, P<0.001.

FIGS. 2A and 2B illustrates the functional analysis of the genes associated with prostatic acinar differentiation. FIG. 2A shows functional clustering of the genes associated with prostatic glandular differentiation. The enriched functional gene categories segregated according to Gene Ontology biological process are depicted as squares with the cross-sectional area representing the number of the genes included in each category. The genes associated with each category are depicted as circles with red indicating an increase and green indicating a decrease in expression levels compared between prostatic acini and cell clusters. FIG. 2B shows fold changes in the transcript levels of the genes associated with epithelial differentiation or the hormonal or secretory functions of prostatic glands in RWPE-1 acini or malignant LNCaP spheroids versus cell clusters as measured by quantitative real time-PCR analyses. Data are represented as mean±SEM. n=3. *, P<0.05; **, P<0.01; ***, P<0.001.

FIG. 3 shows Kaplan-Meier survival curves comparing relapse-free survival of 21 prostate cancer patients in the BWH cohort. The patients were stratified into two groups with high and low r_{acini}. P values were calculated using the log-rank test.

FIG. 4 shows Kaplan-Meier survival curves comparing relapse-free survival of 29 prostate cancer patients in the Lapointe et al. cohort stratified according to r_{acini}. P values were calculated using the log-rank test.

FIG. 5 shows the selection of the 12-gene set based on the distribution of concordance index (C-index) in the prediction of risk of disease relapse in the 21 patients with prostate cancer in the BWH cohort. C-index statistics analysis was conducted using the ‘survcomp’ package in the statistical programming language R (cran.r-project.org).

FIG. 6 shows Kaplan-Meier survival curves comparing relapse-free survival of 21 patients with prostate cancer in the BWH cohort. The patients were stratified into two groups based on predicted risk of relapse based on the recurrence score (Equation 1) calculated according the transcript abundance levels of the 12 molecular markers in

Table. P values were calculated using the log-rank test.

FIG. 7 shows Kaplan-Meier survival curves comparing relapse-free survival of 29 patients with prostate cancer in the Lapointe et al. cohort. The patients were stratified into two groups based on the recurrence score (Equation 1) calculated according to the expression pattern of the 12 molecular markers in

Table. P values were calculated using the log-rank test.

FIG. 8 shows relapse-free survival of 21 patients with prostate cancer in the BWH cohort stratified based on the expression levels of the respective molecular markers in

Table. The threshold value for each gene marker was determined by the maximal Youden's index. P values were calculated using the log-rank test.

FIG. 9 shows representative immunostaining of PDCD4 (i, ii), KLF6 (iii, iv) and ABCG1 (v, vi) in prostate cancer tissues from the CFMC cohort (400× magnification). Shown are tumors with high (i, iii, v) or low (ii, iv, vi) staining intensities of the respective markers.

FIG. 10 shows Kaplan-Meier survival curves comparing recurrence-free survival of 61 prostate cancer patients in the CFMC cohort stratified according to the staining intensities of PDCD4, ABCG1 or KLF6. The staining patterns were quantified using the histological score (H-score). The threshold value for each gene marker was determined by the maximal Youden's index. P values were calculated using the log-rank test.

FIG. 11 shows Kaplan-Meier survival curves comparing recurrence-free survival of 61 prostate cancer patients in the CFMC cohort. The patients were stratified into two groups based on the recurrence score (Equation 1) calculated according to the staining intensities (quantified by H-score) of PDCD4, ABCG1 and KLF6. P values were calculated using the log-rank test.

FIG. 12 shows Kaplan-Meier survival curves comparing recurrence-free survival of 21 prostate cancer patients in the BWH cohort. The patients were stratified into two groups based on the recurrence score (Equation 1) calculated according to the transcript abundance levels, as represented by the probe hybridization intensities, of PDCD4, ABCG1 and KLF6. P values were calculated using the log-rank test.

FIG. 13 shows Kaplan-Meier survival curves comparing recurrence-free survival of 61 prostate cancer patients in the CFMC cohort. The patients were stratified into two groups based on the recurrence score (Equation 1) calculated according to the staining intensities (quantified by H-score) of PDCD4 and ABCG1. P values were calculated using the log-rank test.

FIG. 14 shows Kaplan-Meier survival curves comparing recurrence-free survival of 21 prostate cancer patients in the BWH cohort. The patients were stratified into two groups based on the recurrence score (Equation 1) calculated according to the transcript abundance levels, as represented by the probe hybridization intensities, of PDCD4 and ABCG1. P values were calculated using the log-rank test.

### DETAILED DESCRIPTION OF THE EMBODIMENTS

### Definition

As used herein, “prostate cancer” refers to malignant mammalian cancers, especially adenocarcinomas, derived from prostate epithelial cells. Prostate cancers embraced in the current application include both metastatic and non-metastatic cancers.

The term “differentiation” refers to generalized or specialized changes in structures or functions of an organ or tissue during development. The concept of differentiation is well known in the art and requires no further description herein. For example, differentiation of prostate refers to, among others, the process of glandular structure formation and/or the acquisition of hormonal or secretory functions of normal prostatic glands.

As used herein, the term “clinical prognosis” refers to the outcome of subjects with prostate cancer comprising the likelihood of tumor recurrence, survival, disease progression, and response to treatments. The recurrence of prostate cancer after treatment (e.g., prostatectomy) is indicative of a more aggressive cancer, a shorter survival of the host (e.g., prostate cancer patients), an increased likelihood of an increase in the size, volume or number of tumors, and/or an increased likelihood of failure of treatments.

As used herein, the term “predicting clinical prognosis” refers to providing a prediction of the probable course or outcome of prostate cancer, including prediction of metastasis, multidrug resistance, disease free survival, overall survival, recurrence, etc. The methods can also be used to devise a suitable therapy for cancer treatment, e.g., by indicating whether or not the cancer is still at an early stage or if the cancer had advanced to a stage where aggressive therapy would be ineffective.

As used herein, the term “recurrence” refers to the return of a prostate cancer after an initial or subsequent treatment(s). Representative treatments include any form of surgery (e.g., radical prostatectomy), any form of radiation treatment, any form of chemotherapy or biological therapy, any form of hormone treatment. In some examples, recurrence of the prostate cancer is marked by rising prostate-specific antigen (PSA) to levels (e.g., PSA of at least 0.4 ng/ml or two consecutive PSA values of 0.2 mg/ml and rising) (Stephenson et al., 2006) and/or by identification of prostate cancer cells in any biological sample from a subject with prostate cancer.

As used herein, the term “disease progression” refers to a situation wherein one or more indices of prostate cancer (e.g., serum PSA levels, measurable tumor size or volume, or new lesions) show that the disease is advancing despite treatment(s).

The terms “molecular marker”, “gene marker”, “cancer-associated antigen”, “tumor-specific marker”, “tumor marker”, “maker”, or “biomarker” interchangeably refer to a molecule or a gene (typically protein or nucleic acid such as RNA) that is differentially expressed in the cell, expressed on the surface of a cancer cell or secreted by a cancer cell in comparison to a non-cancer cell or another cancer cells, and which is useful for the diagnosis of cancer, for providing a prognosis, and for preferential targeting of a pharmacological agent to the cancer cell. Oftentimes, a cancer-associated antigen is a molecule that is overexpressed or underexpressed in a cancer cell in comparison to a non-cancer cell or another cancer cells, for instance, 1-fold over expression, 2-fold overexpression, 3-fold overexpression or more in comparison to a non-cancer cell or, for instance, 20%, 30%, 40%, 50% or more underexpressed in comparison to a non-cancer cell. Oftentimes, a cancer-associated antigen is a molecule that is inappropriately synthesized in the cancer cell, for instance, a molecule that contains deletions, additions or mutations in comparison to the molecule expressed in a non-cancer cell. Oftentimes, a cancer-associated antigen will be expressed exclusively on the cell surface of a cancer cell and not synthesized or expressed on the surface of a normal cell. Exemplified cell surface tumor markers include prostate-specific antigen (PSA) for prostate cancer, the proteins c-erbB-2 and human epidermal growth factor receptor (HER) for breast cancer, and carbohydrate mucins in numerous cancers, including breast, ovarian and colorectal. Other times, a cancer-associated antigen will be expressed primarily not on the surface of the cancer cell.

The term “differentially expressed” or “differentially regulated” refers generally to a protein or nucleic acid that is overexpressed (upregulated) or underexpressed (downregulated) in one sample compared to at least one other sample in the context of the present invention.

“ABCG1”, “PDCD4”, “KLF6” and other molecular markers recited herein, including those found in

Table, refer to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 60% nucleotide sequence identity, 65%, 70%, 75%, 80%, 85%, 90%, preferably 91%, 92%, 93%, 94%, 95%, 96%, 97%, 98% or 99% or higher nucleotide sequence identity, preferably over a region of at least about 10, 15, 20, to 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. Truncated and alternatively spliced forms of these antigens are included in the definition.

It will be understood by the skilled artisan that markers may be used singly or in combination with other markers for any of the uses, e.g., diagnosis or prognosis of multidrug resistant cancers, disclosed herein.

“Biological sample” includes sections of tissues such as biopsy and autopsy samples, and frozen sections taken for histologic purposes. Such samples include prostate cancer tissues, blood and blood fractions or products (e.g., serum, plasma, platelets, red blood cells, and the like), sputum, tissue, cultured cells, e.g., primary cultures, explants, and transformed cells, stool, urine, etc.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., breast, etc.), the size and type of the tumor, among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy”, or a “fine-needle aspiration biopsy” which generally obtains a suspension of cells from within a target tissue.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

Exemplary Molecular Markers:

ATP-Binding Cassette, Sub-Family G, Member 1 (ABCG1)

The human ATP-binding cassette, sub-family G, member 1 (ABCG1) gene (NCBI Entrez Gene 9619) is located on chromosome 21 at gene map locus 21q22.3 and encodes a multi-pass membrane protein predominantly localized in the endoplasmic reticulum (ER) and Golgi membranes. Six alternative splice variants have been identified. Exemplary ABCG1 sequences are publically available, for example from GenBank (e.g., accession numbers NM_{—}004915.3, NM_{—}016818.2, NM_{—}207174.1, NM_{—}997510, NM_{—}207628.1, and NM_{—}207629.1 (mRNAs) and NP_{—}004906.3, NP_{—}058198.2, NP_{—}997057.1, NP_{—}997510.1, NP_{—}997511.1, and NP_{—}997512.1 (proteins)), or UniProtKB (e.g., P45844).

Programmed Cell Death 4 (PDCD4)

The human Programmed cell death 4 (PDCD4) gene (NCBI Entrez Gene 27250) is located on chromosome 10 at gene map locus 10q24 and encodes a nuclear and cytoplasmic shuttling protein. Three alternative splice variants have been identified. Exemplary PDCD4 sequences publically available, for example from GenBank (e.g., accession numbers NM_{—}001199492.1, NM_{—}014456.4, and NM_{—}145341.3 (mRNAs), and NP_{—}001186421.1, NP_{—}055271.2, and NP_{—}663314.1 (proteins)), or UniProtKB (e.g., Q53EL6).

Kruppel-Like Factor 6 (KLF6)

The human Kruppel-like factor 6 (KLF6) gene (NCBI Entrez Gene 1316) is located on chromosome 10 at gene map locus 10q15 and encodes a nuclear protein. Three alternative splice variants have been identified. Exemplary KLF6 sequences publically available, for example from GenBank (e.g., accession numbers NM_{—}001160124.1, NM_{—}001160125.1, and NM_{—}001300.5 (mRNAs), and NP_{—}001153596.1, NP_{—}001153597.1, and NP_{—}001291.3 (proteins)), or UniProtKB (e.g., Q99612).

In the present application, the molecular markers comprising the marker genes ABCG1, PDCD4, KLF6, ST6, BTD, BANF1, IRS1, ZNF185, ANXA11, DUSP2, KLF4, DSC2 or any combination thereof is provided to predict clinical prognosis of prostate cancer. A method and a kit based on the above molecular markers are also provided.

Being the molecular marker, the marker genes ABCG1, PDCD4, KLF6, ST6, BTD, BANF1, IRS1, ZNF185, ANXA11, DUSP2, KLF4 and DSC can be used alone or in combination. The molecular marker includes the gene, the RNA transcript, and the expression product (e.g. protein), which can be wild-type, truncated or alternatively spliced forms.

In one embodiment, a combination of at least two of the above marker genes are preferred, such as 3, 4, 5, 6, 7, 8, 9, 10, 11, or all 12 of the marker genes. In a preferred embodiment, the molecular marker is a 12-gene model, using all of the marker genes for prediction. In another preferred embodiment, the molecular marker is a 3-gene model or a 2-gene model, wherein the marker gene is selected from a group consisting of ABCG1, PDCD4 and KLF6. More particularly, the molecular marker is a combination of ABCG1, PDCD4 and KLF6, or a combination of ABCG1 and PDCD4.

The expression level of the marker gene can be determined based on a RNA transcript of the marker gene, or an expression product thereof, or their combination. In one embodiment, the means for detecting the expression level of the marker gene comprises nucleic acid probe, aptamer, antibody, or any combination thereof, which is able to specifically recognize the RNA transcript or the expression product (e.g. protein) of the marker gene. More particularly, the expression level of RNA transcript of a marker gene can be detected by polymerase chain reaction (PCR), northern blotting assay, RNase protection assay, oligonucleotide microarray assay, RNA in situ hybridization and the like, and the expression level of an expression product of a marker gene, such as protein or polypeptide, can be detected by immunoblotting assay, immunohistochemistry, two-dimensional protein electrophoresis, mass spectroscopy analysis assay, histochemistry stain and the like. The above detection means can be used alone or in combination.

The biological sample is defined as above, which can be obtained by aspiration, biopsy, or surgical resection. The biological sample can be fresh, frozen, or formalin fixed paraffin embedded (FFPE) prostate tumor specimens.

In one embodiment, nucleic acid binding molecules such as probes, oligonucleotides, oligonucleotide arrays, and primers can be used in assays to detect differential RNA expression of marker genes in patient samples, e.g., RT-PCR, qPCR and nucleic acid microarrays.

In another embodiment, the detection of protein expression level comprises the use of antibodies specific to the gene markers and immunohistochemistry staining on fixed (e.g., formalin-fixed) and/or wax-embedded (e.g., paraffin-embedded) prostate tumor tissues. The immunohistochemistry methods may be performed manually or in an automated fashion.

In another embodiment, the antibodies or nucleic acid probes can be applied to patient samples immobilized on microscope slides. The resulting antibody staining or in situ hybridization pattern can be visualized using any one of a variety of light or fluorescent microscopic methods known in the art.

In another embodiment, analysis of the protein or nucleic acid can be achieved by such as high pressure liquid chromatography (HPLC), alone or in combination with mass spectrometry (e.g., MALDI/MS, MALDI-TOF/MS, tandem MS, etc.).

In one embodiment, the clinical prognosis includes the likelihood of disease progression, clinical prognosis, recurrence, death and the like. The disease progression comprises such as classification of prostate cancer, determination of differentiation degree of prostate cancer cells and the like.

In another embodiment, the clinical prognosis can be a time interval to between the date of disease diagnosis or surgery and the date of disease recurrence or metastasis; a time interval between the date of disease diagnosis or surgery and the date of death of the subject; at least one of changes in number, size and volume of measurable tumor lesion of prostate cancer; or any combination thereof. Said change of the tumor lesion can be determined by visual, radiological and/or pathological examination of said prostate cancer before and at various time points during and after diagnosis or surgery.

In the present application, the reference level is applied as the baseline of the prediction, which can be determined based on the normalized expression level of the marker gene in a plurity of prostate cancer patients. Typically, the reference level can be a the threshold reference value, which is representative of a polypeptide or polynucleotide of the marker gene in a large number of persons or tissues with prostate cancer and whose clinical prognosis data are available, as measured using a tissue sample or biopsy or other biological sample such a cell, serum or blood. Said threshold reference values are determined by defining levels wherein said subjects whose tumors have expression levels of said markers above said threshold reference level(s) are predicted as having a higher or lower degree of differentiation or risk of poor clinical prognosis or disease progression than those with expression levels below said threshold reference level(s). Variation of levels of a polypeptide or polynucleotide of the invention from the reference range (either up or down) indicates that the patient has a higher or lower degree of differentiation or risk of poor clinical prognosis or disease progression than those with expression levels below said threshold reference level(s).

To compare the expression level of the marker gene and the reference level, statistical methods including, without limitation, class distinction using unsupervised methods (e.g., k-means, hierarchical clustering, principle components, non-negative matrix factorization, or multidimensional scaling) (Hastie et al., 2009), supervised methods (e.g., discriminant analysis, support vector machines, or k-nearest-neighbors) or semi-supervised methods, or outcome prediction (e.g., relapse-free survival, disease progression, or overall survival) using Cox regression model (Kalbfleisch and Prentice, 2002), accelerated failure time model, Bayesian survival model, or smoothing analysis for survival data (Wand, 2003) may be involved.

In one embodiment, comparing with the reference level, the increased expression level of the marker gene indicates an increased likelihood of positive clinical prognosis, such as long-term survival without prostate cancer recurrence. In another embodiment, the increased expression level of the marker gene may indicate an decreased likelihood of positive clinical prognosis, such as recurrence rate of prostate cancer.

In the present application, the kit comprises a means for detecting the expression level of the molecular marker, for example, a probe or an antibody. The kit can further comprise a control group such as a probe or an antibody specifically binding to housekeeping gene(s) or protein(s) (e.g., beta-actin, GAPDH, RPL13A, tubulin, and the likes).

In one preferred embodiment, the kit can include at least one nucleic acid probe specific for ABCG1 transcript, PDCD4 transcript or KLF6 transcript; at least one pair of primers for specific amplification of ABCG1, PDCD4 or KLF6; and/or at least one antibody specific for ABCG1 protein, PDCD4 protein or KLF6 protein. The kit further comprises a nucleic acid probe, primers, and/or an antibody specific for housekeeping gene/transcript/protein.

In one embodiments, the primary detection means (e.g., probe, primers, or antibody) can be directly labeled with a fluorophore, chromophore, or enzyme capable of producing a detectable product (e.g., alkaline phosphates, horseradish peroxidase and others commonly known in the art), or, a secondary detection means such as secondary antibodies or non-antibody hapten-binding molecules (e.g., avidin or streptavidin) can be applied. The secondary detection means can be directly labeled with a detectable moiety. In other instances, the secondary or higher order antibody can be conjugated to a hapten (e.g., biotin, DNP, or FITC), which is detectable by a cognate hapten binding molecule (e.g., streptavidin horseradish peroxidase, streptavidin alkaline phosphatase, or streptavidin QDot™). In another embodiments, the kit can further comprise a colorimetric reagent, which is used in concert with primary, secondary or higher order detection means that are labeled with enzymes for the development of such colorimetric reagents.

In one embodiment, the kit further comprises a positive and/or a negative control sample(s), such as mRNA samples that contain or do not contain transcripts of the marker genes, protein lysates that contain or do not contain proteins or fragmented proteins encoded by the marker genes, and/or cell line or tissue known to express or not express the marker genes.

In some embodiments, the kit may further comprise a carrier, such as a box, a bag, a vial, a tube, a satchel, plastic carton, wrapper, or other container. The components of the kit can be enclosed in a single packing unit, which may have compartments into which one or more components of the kit can be placed; or, the kit includes one or more containers that can retain, for example, one or more biological samples to be tested. In some embodiments, the kit further comprises buffers and other reagents that can be used for the practice the prediction method.

The combination of molecular markers of the present application can be applied to a microarray, such as nucleic acid array or protein array. The microarray comprises a solid surface (e.g., glass slide) upon which the specific binding agents (e.g., cDNA probes, mRNA probes, or antibodies) are immobilized. The specific binding agents are distinctly located in an addressable (e.g., grid) format on the array. The specific binding agents interact with their cognate targets present in the sample. The pattern of binding of targets among all immobilized agents provides a profile of gene expression.

In one embodiment, the microarray consists of binding agents specific for at least two of the marker genes, for example, an microarray consists of nucleic acid probes or antibodies specific for ABCG1, PDCD4 and KLF6. The microarray can further includes nucleic acid probes or antibodies specific for one or a plurality of housekeeping genes or gene products, such as mRNA, cDNA or protein.

The nucleic acid probes or antibodies forming the array can be directly linked to the support or attached to the support by oligonucleotides or other molecules that serve as spacers or linkers to the solid support. The solid support can be glass slides or formed from an organic polymer. A variety of array formats can be employed in accordance with the present application. For instance, a linear array of oligonucleotide bands, a two-dimensional pattern of discrete cells, and the like.

The following examples are given for illustrative purposes only and are not intended to be limiting unless otherwise specified. It should be appreciated by those of skill in the art that the techniques disclosed in the examples which follow represent techniques discovered by the inventors to function well in the practice of invention, and thus can be considered to constitute preferred modes for its practice. Those of skill in the art should appreciate that many changes can be made in the specific embodiments which are disclosed and still obtain a like or similar result without departing from the spirit and scope of the invention.

### EXAMPLES

### Example 1

### Identification of the Gene Expression Profile Associated with Differentiation of Prostatic Acini

The acinar differentiation process of prostatic glands was recapitulated by culturing prostatic epithelial RWPE-1 cells (Bello et al., 1997) within a physiological relevant three-dimensional (3D) culture model, as described before (Weaver et al., 1997). RWPE-1 cells were immortalized prostate epithelial cells derived from human prostate acini and were known to retain normal cytogenetic and functional characteristics (Bello et al., 1997). RWPE-1 cells were embedded and grown within a thick layer of 3D reconstituted basement membrane gel (Matrigel, BD Biosciences). The culture was maintained in Keratinocyte-SFM (Sigma-Aldrich) supplemented with bovine pituitary extract, 10 ng/ml epidermal growth factor and antibiotics (all from to Invitrogen) (Bello et al., 1997; Liu et al., 1998).

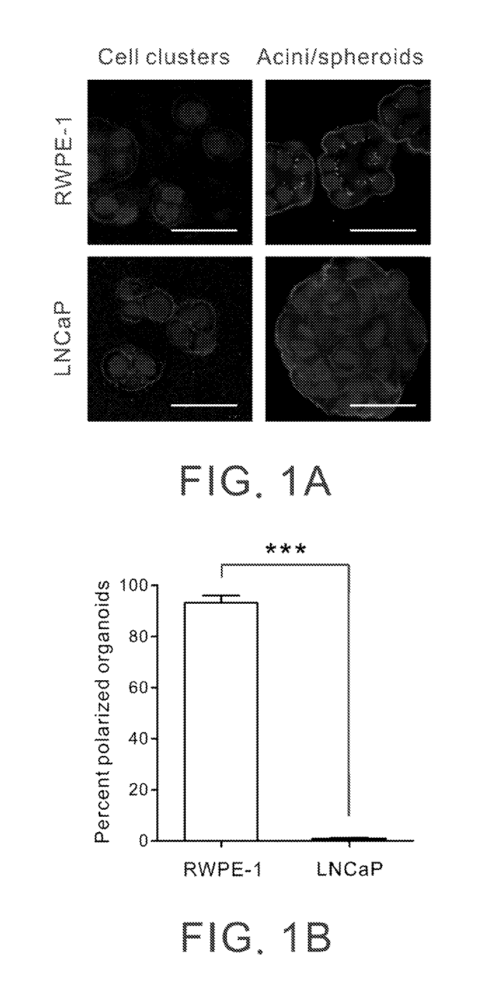

As shown in FIGS. 1A and 1B, when cultured within such a context for a short duration (48 hours), RWPE-1 cells formed small cell clusters lacking cell polarization or tissue architectures. Following a prolonged length of time in 3D culture (10-12 days), a considerable proportion (average 93.1%) of these cells underwent morphological organization, resulting in the formation of round, acini-like structures reminiscent of normal prostatic glands or low-grade PCA. Confocal image analysis confirmed that these structures were composed of a single layer of cells with apico-basal polarization, as indicated by the location of the basal surface marker α6-integrin (red) and the apical marker GM130 (green), that surrounded a hollow central lumen (FIG. 1A). Examination of the 3D structures revealed that up to 93.1% of RWPE-1 cells formed polarized acini while very few of prostate carcinoma LNCaP cells were capable of forming polarized architectures (FIG. 1B).

To dissect the gene expression alterations related to this prostatic acinar differentiation process, global gene expression profiling experiments was carried out on RWPE-1 cells clusters formed in early-stage culture and acini formed at latter stages. Briefly, total RNA samples were extracted using TRIZOL (Invitrogen) and then purified using a RNeasy mini-kit and a DNase treatment (Qiagen). Experiments were performed in triplicate. Gene expression analysis was performed on an Affymetrix Human Genome U133A 2.0 Plus GeneChip platform according to the manufacturer's protocol (Affymetrix). The hybridization intensity data was processed using the GeneChip Operating software (Affymetrix) and the genes were filtered based on the Affymetrix P/A/M flags to retain the genes that were present in at least three of the replicate samples in at least one of the culture conditions. To select differentially expressed genes within a comparison group, a false discovery rate less than 0.025 was used.

Table 1 provides a detailed list of 411 unique genes (represented by 447 Affymetrix probe sets) were identified as differential expression genes during the acinar differentiation of RWPE-1 cells. These genes were identified from the microarray experiments based on their expression levels significantly different between RWPE-1 cell clusters and acini. The genes are ranked in descending order according to the ratio between the mean hybridization intensity of each probe in RWPE-1 acini and that in RWPE-1 cell clusters.

TABLE 1
The 411 genes (represented by 447 Affymetrix probe sets) that were
differentially expressed in RWPE-1 acini (A) and cell clusters (C)

| Expression | | | | |
|---|---|---|---|---|
| ratio | Affymetrix | Gene | ENTREZ | |
| (A vs. C) | probe set ID | symbol | Gene ID | Gene title |
| 79.53 | 231771_at | GJB6 | 10804 | gap junction protein, beta 6, 30 kDa |
| 49.21 | 206276_at | LY6D | 8581 | lymphocyte antigen 6 complex, locus D |
| 26.71 | 201150_s_at | TIMP3 | 7078 | TIMP metallopeptidase inhibitor 3 |
| 24.71 | 201313_at | ENO2 | 2026 | enolase 2 (gamma, neuronal) |
| 24.39 | 213075_at | OLFML2 | 169611 | olfactomedin-like 2A |
| | | A | | |
| 21.38 | 232082_x_at | SPRR3 | 6707 | small proline-rich protein 3 |
| 18.05 | 205064_at | SPRR1B | 6699 | small proline-rich protein 1B (cornifin) |
| 17.84 | 202859_x_at | IL8 | 3576 | interleukin 8 |
| 17.82 | 206125_s_at | KLK8 | 11202 | kallikrein-related peptidase 8 |
| 17.39 | 209732_at | CLEC2B | 9976 | C-type lectin domain family 2, member B |
| 15.53 | 215184_at | DAPK2 | 23604 | death-associated protein kinase 2 |
| 14.52 | 201147_s_at | TIMP3 | 7078 | TIMP metallopeptidase inhibitor 3 |
| 14.47 | 204130_at | HSD11B2 | 3291 | hydroxysteroid (11-beta) dehydrogenase 2 |
| 14.07 | 200632_s_at | NDRG1 | 10397 | N-myc downstream regulated gene 1 |
| 13.31 | 219995_s_at | ZNF750 | 79755 | zinc finger protein 750 |
| 13.27 | 212531_at | LCN2 | 3934 | lipocalin 2 |
| 13.09 | 214549_x_at | SPRR1A | 6698 | small proline-rich protein 1A |
| 12.35 | 202748_at | GBP2 | 2634 | guanylate binding protein 2, |
| | | | | interferon-inducible |
| 11.21 | 209720_s_at | SERPINB | 6317 | serpin peptidase inhibitor, clade B |
| | | 3 | | (ovalbumin), member 3 |
| 11.05 | 202917_s_at | S100A8 | 6279 | S100 calcium binding protein A8 |
| 10.76 | 213693_s_at | MUC1 | 4582 | mucin 1, cell surface associated |
| 10.3 | 210413_x_at | SERPINB | 6317 /// | serpin peptidase inhibitor, clade B |
| | | 3 /// | 6318 | (ovalbumin), member 3 /// serpin peptidase |
| | | SERPINB | | inhibitor, clade B (ovalbumin), member 4 |
| | | 4 | | |
| 9.58 | 208607_s_at | SAA1 /// | 6288 /// | serum amyloid A1 /// serum amyloid A2 |
| | | SAA2 | 6289 | |
| 9.53 | 224009_x_at | DHRS9 | 10170 | dehydrogenase/reductase (SDR family) |
| | | | | member 9 |
| 9.42 | 206008_at | TGM1 | 7051 | transglutaminase 1 (K polypeptide epidermal |
| | | | | type I, |
| | | | | protein-glutamine-gamma-glutamyltransferase) |
| 9.12 | 209230_s_at | NUPR1 | 26471 | nuclear protein 1 |
| 9.11 | 218960_at | TMPRSS4 | 56649 | transmembrane protease, serine 4 |
| 9.05 | 212706_at | LOC10028 | 1001322 | RAS p21 protein activator 4 pseudogene /// |
| | | 6937 /// | 14 /// | similar to HSPC047 protein /// similar to |
| | | LOC10028 | 1001330 | RAS p21 protein activator 4 /// similar to |
| | | 7164 /// | 05 /// | HSPC047 protein /// RAS p21 protein |
| | | RASA4 | 1001347 | activator 4 |
| | | | 22 /// | |
| | | | 10156 /// | |
| | | | 401331 | |
| 8.99 | 209719_x_at | SERPINB | 6317 | serpin peptidase inhibitor, clade B |
| | | 3 | | (ovalbumin), member 3 |
| 8.76 | 201149_s_at | TIMP3 | 7078 | TIMP metallopeptidase inhibitor 3 |
| 8.71 | 230323_s_at | TMEM45 | 120224 | transmembrane protein 45B |
| | | B | | |
| 7.73 | 223278_at | GJB2 | 2706 | gap junction protein, beta 2, 26 kDa |
| 7.61 | 204734_at | KRT15 | 3866 | keratin 15 |
| 7.58 | 209800_at | KRT16 | 3868 | keratin 16 |
| 7.35 | 219799_s_at | DHRS9 | 10170 | dehydrogenase/reductase (SDR family) |
| | | | | member 9 |
| 7.28 | 213240_s_at | KRT4 | 3851 | keratin 4 |
| 7.24 | 213293_s_at | TRIM22 | 10346 | tripartite motif-containing 22 |
| 7.22 | 201141_at | GPNMB | 10457 | glycoprotein (transmembrane) nmb |
| 7.13 | 237465_at | USP53 | 54532 | ubiquitin specific peptidase 53 |
| 6.66 | 236225_at | GGT6 | 124975 | gamma-glutamyltransferase 6 |
| 6.56 | 205158_at | RNASE4 | 6038 | ribonuclease, RNase A family, 4 |
| 6.43 | 223484_at | C15orf48 | 84419 | chromosome 15 open reading frame 48 |
| 6.33 | 226403_at | TMC4 | 147798 | transmembrane channel-like 4 |
| 6.17 | 217528_at | CLCA2 | 9635 | CLCA family member 2, chloride channel |
| | | | | regulator |
| 6.13 | 204351_at | S100P | 6286 | S100 calcium binding protein P |
| 6.05 | 226388_at | TCEA3 | 6920 | transcription elongation factor A (SII), 3 |
| 6.01 | 228640_at | PCDH7 | 5099 | protocadherin 7 |
| 6 | 219232_s_at | EGLN3 | 112399 | egl nine homolog 3 (C. elegans) |
| 5.94 | 203438_at | STC2 | 8614 | stanniocalcin 2 |
| 5.86 | 204985_s_at | TRAPPC6 | 79090 | trafficking protein particle complex 6A |
| | | A | | |
| 5.68 | 218537_at | HCFC1R1 | 54985 | host cell factor C1 regulator 1 (XPO1 |
| | | | | dependent) |
| 5.18 | 217767_at | C3 | 718 | complement component 3 |
| 5.18 | 216379_x_at | CD24 | 1001339 | CD24 molecule |
| | | | 41 | |
| 5.13 | 231577_s_at | GBP1 | 2633 | guanylate binding protein 1, |
| | | | | interferon-inducible, 67 kDa |
| 5.11 | 202269_x_at | GBP1 | 2633 | guanylate binding protein 1, |
| | | | | interferon-inducible, 67 kDa |
| 5.05 | 210046_s_at | IDH2 | 3418 | isocitrate dehydrogenase 2 (NADP+), |
| | | | | mitochondrial |
| 5.02 | 204542_at | ST6GALN | 10610 | ST6 |
| | | AC2 | | (alpha-N-acetyl-neuraminyl-2,3-beta-galactosyl- |
| | | | | 1,3)-N-acetylgalactosaminide |
| | | | | alpha-2,6-sialyltransferase 2 |
| 4.99 | 238689_at | GPR110 | 266977 | G protein-coupled receptor 110 |
| 4.98 | 214598_at | CLDN8 | 9073 | claudin 8 |
| 4.95 | 201008_s_at | TXNIP | 10628 | thioredoxin interacting protein |
| 4.86 | 212143_s_at | IGFBP3 | 3486 | insulin-like growth factor binding protein 3 |
| 4.78 | 231929_at | IKZF2 | 22807 | IKAROS family zinc finger 2 (Helios) |
| 4.71 | 209771_x_at | CD24 | 1001339 | CD24 molecule |
| | | | 41 | |
| 4.68 | 213988_s_at | SAT1 | 6303 | spermidine/spermine N1-acetyltransferase 1 |
| 4.54 | 266_s_at | CD24 | 1001339 | CD24 molecule |
| | | | 41 | |
| 4.49 | 210095_s_at | IGFBP3 | 3486 | insulin-like growth factor binding protein 3 |
| 4.47 | 203126_at | IMPA2 | 3613 | inositol(myo)-1(or 4)-monophosphatase 2 |
| 4.4 | 203758_at | CTSO | 1519 | cathepsin O |
| 4.39 | 201010_s_at | TXNIP | 10628 | thioredoxin interacting protein |
| 4.38 | 204567_s_at | ABCG1 | 9619 | ATP-binding cassette, sub-family G |
| | | | | (WHITE), member 1 |
| 4.36 | 208650_s_at | CD24 | 1001339 | CD24 molecule |
| | | | 41 | |
| 4.3 | 217272_s_at | SERPINB | 5275 | serpin peptidase inhibitor, clade B |
| | | 13 | | (ovalbumin), member 13 |
| 4.25 | 202022_at | ALDOC | 230 | aldolase C, fructose-bisphosphate |
| 4.23 | 204379_s_at | FGFR3 | 2261 | fibroblast growth factor receptor 3 |
| 4.19 | 239430_at | IGFL1 | 374918 | IGF-like family member 1 |
| 4.19 | 1558846_at | PNLIPRP3 | 119548 | pancreatic lipase-related protein 3 |
| 4.08 | 200696_s_at | GSN | 2934 | gelsolin (amyloidosis, Finnish type) |
| 4.02 | 230188_at | NIPAL4 | 348938 | ichthyin protein |
| 4.02 | 213750_at | RSL1D1 | 26156 | ribosomal L1 domain containing 1 |
| 3.96 | 228002_at | IDI2 | 91734 | isopentenyl-diphosphate delta isomerase 2 |
| 3.95 | 202086_at | MX1 | 4599 | myxovirus (influenza virus) resistance 1, |
| | | | | interferon-inducible protein p78 (mouse) |
| 3.83 | 236055_at | DQX1 | 165545 | DEAQ box polypeptide 1 (RNA-dependent |
| | | | | ATPase) |
| 3.8 | 236009_at | PERP | — | — |
| 3.79 | 208651_x_at | CD24 | 1001339 | CD24 molecule |
| | | | 41 | |
| 3.75 | 225283_at | ARRDC4 | 91947 | arrestin domain containing 4 |
| 3.71 | 220120_s_at | EPB41L4 | 64097 | erythrocyte membrane protein band 4.1 like |
| | | A | | 4A |
| 3.7 | 224701_at | PARP14 | 54625 | poly (ADP-ribose) polymerase family, |
| | | | | member 14 |
| 3.68 | 207543_s_at | P4HA1 | 5033 | procollagen-proline, 2-oxoglutarate |
| | | | | 4-dioxygenase (proline 4-hydroxylase), alpha |
| | | | | polypeptide 1 |
| 3.65 | 208960_s_at | KLF6 | 1316 | Kruppel-like factor 6 |
| 3.65 | 201565_s_at | ID2 | 3398 | inhibitor of DNA binding 2, dominant |
| | | | | negative helix-loop-helix protein |
| 3.6 | 229414_at | PITPNC1 | 26207 | phosphatidylinositol transfer protein, |
| | | | | cytoplasmic 1 |
| 3.56 | 213895_at | EMP1 | 2012 | epithelial membrane protein 1 |
| 3.53 | 207076_s_at | ASS1 | 445 | argininosuccinate synthetase 1 |
| 3.53 | 201009_s_at | TXNIP | 10628 | thioredoxin interacting protein |
| 3.5 | 220370_s_at | USP36 | 57602 | ubiquitin specific peptidase 36 |
| 3.49 | 224657_at | ERRFI1 | 54206 | ERBB receptor feedback inhibitor 1 |
| 3.46 | 221478_at | BNIP3L | 665 | BCL2/adenovirus E1B 19 kDa interacting |
| | | | | protein 3-like |
| 3.44 | 214696_at | C17orf91 | 84981 | chromosome 17 open reading frame 91 |
| 3.4 | 205476_at | CCL20 | 6364 | chemokine (C-C motif) ligand 20 |
| 3.35 | 221841_s_at | KLF4 | 9314 | Kruppel-like factor 4 (gut) |
| 3.34 | 210592_s_at | SAT1 | 6303 | spermidine/spermine N1-acetyltransferase 1 |
| 3.33 | 219704_at | YBX2 | 51087 | Y box binding protein 2 |
| 3.29 | 1554037_a_at | ZBTB24 | 9841 | zinc finger and BTB domain containing 24 |
| 3.27 | 202207_at | ARL4C | 10123 | ADP-ribosylation factor-like 4C |
| 3.25 | 202331_at | BCKDHA | 593 | branched chain keto acid dehydrogenase E1, |
| | | | | alpha polypeptide |
| 3.22 | 235677_at | SRR | 63826 | Serine racemase |
| 3.2 | 217783_s_at | YPEL5 | 51646 | yippee-like 5 (Drosophila) |
| 3.15 | 206043_s_at | ATP2C2 | 9914 | ATPase, Ca++ transporting, type 2C, |
| | | | | member 2 |
| 3.15 | 208498_s_at | AMY1A | 276 /// | amylase, alpha 1A (salivary) /// amylase, |
| | | /// AMY1B | 277 /// | alpha 1B (salivary) /// amylase, alpha 1C |
| | | /// AMY1C | 278 /// | (salivary) /// amylase, alpha 2A (pancreatic) |
| | | /// | 279 /// | /// amylase, alpha 2B (pancreatic) |
| | | AMY2A | 280 | |
| | | /// AMY2B | | |
| 3.14 | 212580_at | ERAP1 | 51752 | Endoplasmic reticulum aminopeptidase 1 |
| 3.08 | 201860_s_at | PLAT | 5327 | plasminogen activator, tissue |
| 3.08 | 203455_s_at | SAT1 | 6303 | spermidine/spermine N1-acetyltransferase 1 |
| 3.03 | 1554897_s_at | RHBDL2 | 54933 | rhomboid, veinlet-like 2 (Drosophila) |
| 3.03 | 233565_s_at | SDCBP2 | 27111 | syndecan binding protein (syntenin) 2 |
| 3.02 | 202206_at | ARL4C | 10123 | ADP-ribosylation factor-like 4C |
| 2.99 | 228727_at | ANXA11 | 311 | annexin A11 |
| 2.96 | 227642_at | TFCP2L1 | 29842 | Transcription factor CP2-like 1 |
| 2.96 | 222162_s_at | ADAMTS | 9510 | ADAM metallopeptidase with |
| | | 1 | | thrombospondin type 1 motif, 1 |
| 2.95 | 228823_at | POLR2J2 | 84820 | polymerase (RNA) II (DNA directed) |
| | | | | polypeptide J4, pseudogene |
| 2.94 | 203232_s_at | ATXN1 | 6310 | ataxin 1 |
| 2.92 | 226847_at | FST | 10468 | follistatin |
| 2.89 | 201041_s_at | DUSP1 | 1843 | dual specificity phosphatase 1 |
| 2.88 | 212907_at | SLC30A1 | 7779 | Solute carrier family 30 (zinc transporter), |
| | | | | member 1 |
| 2.87 | 226482_s_at | TSTD1 | 1001311 | hypothetical protein LOC100134860 /// KAT |
| | | | 87 /// | protein |
| | | | 1001348 | |
| | | | 60 | |
| 2.86 | 45714_at | HCFC1R1 | 54985 | host cell factor C1 regulator 1 (XPO1 |
| | | | | dependent) |
| 2.86 | 202644_s_at | TNFAIP3 | 7128 | tumor necrosis factor, alpha-induced protein |
| | | | | 3 |
| 2.82 | 200884_at | CKB | 1152 | creatine kinase, brain |
| 2.82 | 239586_at | FAM83A | 84985 | family with sequence similarity 83, member |
| | | | | A |
| 2.82 | 203882_at | IRF9 | 10379 | interferon regulatory factor 9 |
| 2.82 | 202659_at | PSMB10 | 5699 | proteasome (prosome, macropain) subunit, |
| | | | | beta type, 10 |
| 2.8 | 204948_s_at | FST | 10468 | follistatin |
| 2.8 | 238741_at | FAM83A | 84985 | family with sequence similarity 83, member |
| | | | | A |
| 2.8 | 205466_s_at | HS3ST1 | 9957 | heparan sulfate (glucosamine) |
| | | | | 3-O-sulfotransferase 1 |
| 2.8 | 229465_s_at | PTPRS | — | — |
| 2.79 | 91826_at | EPS8L1 | 54869 | EPS8-like 1 |
| 2.77 | 204794_at | DUSP2 | 1844 | dual specificity phosphatase 2 |
| 2.76 | 200768_s_at | MAT2A | 4144 | methionine adenosyltransferase II, alpha |
| 2.73 | 209301_at | CA2 | 760 | carbonic anhydrase II |
| 2.73 | 203585_at | ZNF185 | 7739 | zinc finger protein 185 (LIM domain) |
| 2.71 | 219476_at | C1orf116 | 79098 | chromosome 1 open reading frame 116 |
| 2.7 | 221479_s_at | BNIP3L | 665 | BCL2/adenovirus E1B 19 kDa interacting |
| | | | | protein 3-like |
| 2.7 | 204435_at | NUPL1 | 9818 | nucleoporin like 1 |
| 2.66 | 39249_at | AQP3 | 360 | aquaporin 3 (Gill blood group) |
| 2.66 | 241869_at | APOL6 | 80830 | apolipoprotein L, 6 |
| 2.62 | 213848_at | DUSP7 | — | — |
| 2.6 | 243386_at | CASZ1 | 54897 | castor zinc finger 1 |
| 2.6 | 205014_at | FGFBP1 | 9982 | fibroblast growth factor binding protein 1 |
| 2.59 | 211862_x_at | CFLAR | 8837 | CASP8 and FADD-like apoptosis regulator |
| 2.57 | 208078_s_at | SIK1 | 150094 | SNF1-like kinase |
| 2.57 | 207826_s_at | ID3 | 3399 | inhibitor of DNA binding 3, dominant |
| | | | | negative helix-loop-helix protein |
| 2.57 | 227180_at | ELOVL7 | 79993 | ELOVL family member 7, elongation of long |
| | | | | chain fatty acids (yeast) |
| 2.54 | 218844_at | ACSF2 | 80221 | acyl-CoA synthetase family member 2 |
| 2.54 | 218280_x_at | HIST2H2 | 723790 | histone cluster 2, H2aa3 /// histone cluster 2, |
| | | AA3 /// | /// 8337 | H2aa4 |
| | | HIST2H2 | | |
| | | AA4 | | |
| 2.54 | 200670_at | XBP1 | 7494 | X-box binding protein 1 |
| 2.53 | 228975_at | SP6 | 80320 | Sp6 transcription factor |
| 2.53 | 205660_at | OASL | 8638 | 2′-5′-oligoadenylate synthetase-like |
| 2.48 | 212992_at | AHNAK2 | 113146 | AHNAK nucleoprotein 2 |
| 2.47 | 38037_at | HBEGF | 1839 | heparin-binding EGF-like growth factor |
| 2.46 | 229741_at | MAVS | 57506 | virus-induced signaling adapter |
| 2.46 | 204646_at | DPYD | 1806 | dihydropyrimidine dehydrogenase |
| 2.45 | 202284_s_at | CDKN1A | 1026 | cyclin-dependent kinase inhibitor 1A (p21, |
| | | | | Cip1) |
| 2.44 | 203186_s_at | S100A4 | 6275 | S100 calcium binding protein A4 |
| 2.44 | 225606_at | BCL2L11 | 10018 | BCL2-like 11 (apoptosis facilitator) |
| 2.43 | 37408_at | MRC2 | 9902 | mannose receptor, C type 2 |
| 2.42 | 206166_s_at | CLCA2 | 9635 | CLCA family member 2, chloride channel |
| | | | | regulator |
| 2.39 | 227944_at | PTPN3 | 5774 | protein tyrosine phosphatase, non-receptor |
| | | | | type 3 |
| 2.37 | 202073_at | OPTN | 10133 | optineurin |
| 2.35 | 224558_s_at | MALAT1 | 378938 | metastasis associated lung adenocarcinoma |
| | | | | transcript 1 (non-protein coding) |
| 2.32 | 210793_s_at | NUP98 | 4928 | nucleoporin 98 kDa |
| 2.31 | 202180_s_at | MVP | 9961 | major vault protein |
| 2.31 | 229851_s_at | C11orf54 | 28970 | chromosome 11 open reading frame 54 |
| 2.31 | 238028_at | C6orf132 | 1001289 | hypothetical protein LOC100128918 |
| | | | 18 | |
| 2.3 | 215812_s_at | LOC65356 | 386757 | hypothetical LOC653562 /// solute carrier |
| | | 2 /// | /// 6535 | family 6 (neurotransmitter transporter, |
| | | SLC6A10 | /// | creatine), member 10 (pseudogene) /// solute |
| | | P /// | 653562 | carrier family 6 (neurotransmitter transporter, |
| | | SLC6A8 | | creatine), member 8 |
| 2.29 | 209588_at | EPHB2 | 2048 | EPH receptor B2 |
| 2.26 | 209260_at | SFN | 2810 | stratifin |
| 2.24 | 1555832_s_at | KLF6 | 1316 | Kruppel-like factor 6 |
| 2.23 | 204981_at | SLC22A18 | 5002 | solute carrier family 22, member 18 |
| 2.22 | 226817_at | DSC2 | 1824 | desmocollin 2 |
| 2.22 | 227001_at | NIPAL2 | 79815 | NIPA-like domain containing 2 |
| 2.22 | 201601_x_at | IFITM1 | 8519 | interferon induced transmembrane protein 1 |
| | | | | (9-27) |
| 2.2 | 213455_at | FAM114A | 92689 | family with sequence similarity 114, member |
| | | 1 | | A1 |
| 2.2 | 214290_s_at | HIST2H2 | 723790 | histone cluster 2, H2aa3 /// histone cluster 2, |
| | | AA3 /// | /// 8337 | H2aa4 |
| | | HIST2H2 | | |
| | | AA4 | | |
| 2.19 | 207850_at | CXCL3 | 2921 | chemokine (C-X-C motif) ligand 3 |
| 2.17 | 215001_s_at | GLUL | 2752 | glutamate-ammonia ligase (glutamine |
| | | | | synthetase) |
| 2.16 | 203037_s_at | MTSS1 | 9788 | metastasis suppressor 1 |
| 2.16 | 202431_s_at | MYC | 4609 | v-myc myelocytomatosis viral oncogene |
| | | | | homolog (avian) |
| 2.15 | 227475_at | FOXQ1 | 94234 | forkhead box Q1 |
| 2.15 | 202733_at | P4HA2 | 8974 | procollagen-proline, 2-oxoglutarate |
| | | | | 4-dioxygenase (proline 4-hydroxylase), alpha |
| | | | | polypeptide II |
| 2.14 | 220251_at | C1orf107 | 27042 | chromosome 1 open reading frame 107 |
| 2.13 | 238607_at | ZNF296 | 162979 | zinc finger protein 296 |
| 2.13 | 213223_at | RPL28 | 6158 | ribosomal protein L28 |
| 2.13 | 202794_at | INPP1 | 3628 | inositol polyphosphate-1-phosphatase |
| 2.13 | 202744_at | SLC20A2 | 6575 | solute carrier family 20 (phosphate |
| | | | | transporter), member 2 |
| 2.06 | 229276_at | IGSF9 | 57549 | immunoglobulin superfamily, member 9 |
| 2.05 | 221234_s_at | BACH2 | 60468 | BTB and CNC homology 1, basic leucine |
| | | | | zipper transcription factor 2 |
| 2.04 | 231931_at | PRDM15 | 63977 | PR domain containing 15 |
| 2.03 | 1561723_at | LOC33989 | 339894 | hypothetical protein LOC339894 |
| | | 4 | | |
| 2.02 | 223434_at | GBP3 | 2635 | guanylate binding protein 3 |
| 1.98 | 200732_s_at | PTP4A1 | 7803 | protein tyrosine phosphatase type IVA, |
| | | | | member 1 |
| 1.98 | 207565_s_at | MR1 | 3140 | major histocompatibility complex, class |
| | | | | I-related |
| 1.88 | 225673_at | MYADM | 91663 | myeloid-associated differentiation marker |
| 1.88 | 222668_at | KCTD15 | 79047 | potassium channel tetramerisation domain |
| | | | | containing 15 |
| 1.86 | 225245_x_at | H2AFJ | 55766 | H2A histone family, member J |
| 1.85 | 202071_at | SDC4 | 6385 | syndecan 4 |
| 1.85 | 225198_at | VAPA | 9218 | VAMP (vesicle-associated membrane |
| | | | | protein)-associated protein A, 33 kDa |
| 1.83 | 208308_s_at | GPI | 1001339 | glucose phosphate isomerase /// similar to |
| | | | 51 /// | Glucose phosphate isomerase |
| | | | 2821 | |
| 1.83 | 205047_s_at | ASNS | 440 | asparagine synthetase |
| 1.81 | 230031_at | HSPA5 | 3309 | heat shock 70 kDa protein 5 |
| | | | | (glucose-regulated protein, 78 kDa) |
| 1.8 | 218319_at | PELI1 | 57162 | pellino homolog 1 (Drosophila) |
| 1.79 | 235020_at | TAF4B | 6875 | TAF4b RNA polymerase II, TATA box |
| | | | | binding protein (TBP)-associated factor, |
| | | | | 105 kDa |
| 1.78 | 229292_at | EPB41L5 | 57669 | erythrocyte membrane protein band 4.1 like 5 |
| 1.78 | 202345_s_at | FABP5 | 2171 /// | fatty acid binding protein 5 |
| | | | 728641 | (psoriasis-associated) /// fatty acid binding |
| | | | /// | protein 5-like 2 /// fatty acid binding protein |
| | | | 729163 | 5-like 7 |
| 1.77 | 225339_at | SPAG9 | 9043 | sperm associated antigen 9 |
| 1.77 | 209222_s_at | OSBPL2 | 9885 | oxysterol binding protein-like 2 |
| 1.75 | 201250_s_at | SLC2A1 | 6513 | solute carrier family 2 (facilitated glucose |
| | | | | transporter), member 1 |
| 1.75 | 204686_at | IRS1 | 3667 | insulin receptor substrate 1 |
| 1.74 | 212399_s_at | VGLL4 | 9686 | vestigial like 4 (Drosophila) |
| 1.73 | 210986_s_at | TPM1 | 7168 | tropomyosin 1 (alpha) |
| 1.71 | 212593_s_at | PDCD4 | 27250 | programmed cell death 4 (neoplastic |
| | | | | transformation inhibitor) |
| 1.7 | 1007_s_at | DDR1 | 780 | discoidin domain receptor tyrosine kinase 1 |
| 1.68 | 203409_at | DDB2 | 1643 | damage-specific DNA binding protein 2, |
| | | | | 48 kDa |
| 1.68 | 209270_at | LAMB3 | 3914 | laminin, beta 3 |
| 1.67 | 1560587_s_at | PRDX5 | 25824 | peroxiredoxin 5 |
| 1.66 | 236262_at | MMRN2 | 79812 | multimerin 2 |
| 1.63 | 210749_x_at | DDR1 | 780 | discoidin domain receptor tyrosine kinase 1 |
| 1.62 | 238675_x_at | BTF3L4 | 91408 | basic transcription factor 3-like 4 |
| 1.61 | 214116_at | BTD | 686 | biotinidase |
| 1.61 | 205490_x_at | GJB3 | 2707 | gap junction protein, beta 3, 31 kDa |
| 1.6 | 203117_s_at | PAN2 | 9924 | PAN2 polyA specific ribonuclease subunit |
| | | | | homolog (S. cerevisiae) |
| 1.53 | 205241_at | SCO2 | 9997 | SCO cytochrome oxidase deficient homolog |
| | | | | 2 (yeast) |
| 1.51 | 201142_at | EIF2S1 | 1965 | eukaryotic translation initiation factor 2, |
| | | | | subunit 1 alpha, 35 kDa |
| 1.51 | 213198_at | ACVR1B | 91 | activin A receptor, type IB |
| 1.46 | 236172_at | LTB4R | 1241 | leukotriene B4 receptor |
| 1.26 | 226744_at | METT10D | 79066 | methyltransferase 10 domain containing |
| 0.77 | 204989_s_at | ITGB4 | 3691 | integrin, beta 4 |
| 0.76 | 226361_at | TMEM42 | 131616 | transmembrane protein 42 |
| 0.74 | 207507_s_at | ATP5G3 | 518 | ATP synthase, H+ transporting, |
| | | | | mitochondrial F0 complex, subunit C3 |
| | | | | (subunit 9) |
| 0.74 | 202785_at | NDUFA7 | 4701 | NADH dehydrogenase (ubiquinone) 1 alpha |
| | | | | subcomplex, 7, 14.5 kDa |
| 0.73 | 222992_s_at | NDUFB9 | 4715 | NADH dehydrogenase (ubiquinone) 1 beta |
| | | | | subcomplex, 9, 22 kDa |
| 0.73 | 215765_at | LRRC41 | 10489 | leucine rich repeat containing 41 |
| 0.72 | 218680_x_at | C15orf63 | 25764 | Huntingtin interacting protein K |
| | | /// SERF2 | | |
| 0.7 | 1553987_at | C12orf47 | 51275 | chromosome 12 open reading frame 47 |
| 0.69 | 219219_at | TMEM160 | 54958 | transmembrane protein 160 |
| 0.68 | 244569_at | C8orf37 | 157657 | chromosome 8 open reading frame 37 |
| 0.66 | 220094_s_at | CCDC90A | 63933 | coiled-coil domain containing 90A |
| 0.65 | 218046_s_at | MRPS16 | 51021 | mitochondrial ribosomal protein S16 |
| 0.65 | 223113_at | TMEM138 | 51524 | transmembrane protein 138 |
| 0.65 | 205967_at | HIST1H4 | 121504 | histone cluster 1, H4a /// histone cluster 1, |
| | | C | /// | H4b /// histone cluster 1, H4c /// histone |
| | | | 554313 | cluster 1, H4d /// histone cluster 1, H4e /// |
| | | | /// 8294 | histone cluster 1, H4f /// histone cluster 1, |
| | | | /// 8359 | H4h /// histone cluster 1, H4i /// histone |
| | | | /// 8360 | cluster 1, H4j /// histone cluster 1, H4k /// |
| | | | /// 8361 | histone cluster 1, H4l /// histone cluster 2, |
| | | | /// 8362 | H4a /// histone cluster 2, H4b /// histone |
| | | | /// 8363 | cluster 4, H4 |
| | | | /// 8364 | |
| | | | /// 8365 | |
| | | | /// 8366 | |
| | | | /// 8367 | |
| | | | /// 8368 | |
| | | | /// 8370 | |
| 0.64 | 218685_s_at | SMUG1 | 23583 | single-strand-selective monofunctional |
| | | | | uracil-DNA glycosylase 1 |
| 0.64 | 227522_at | CMBL | 134147 | carboxymethylenebutenolidase homolog |
| | | | | (Pseudomonas) |
| 0.63 | 218381_s_at | U2AF2 | 11338 | U2 small nuclear RNA auxiliary factor 2 |
| 0.63 | 225359_at | DNAJC19 | 131118 | DnaJ (Hsp40) homolog, subfamily C, |
| | | | | member 19 |
| 0.62 | 222116_s_at | TBC1D16 | 125058 | TBC1 domain family, member 16 |
| 0.62 | 219084_at | NSD1 | 64324 | nuclear receptor binding SET domain protein |
| | | | | 1 |
| 0.62 | 209104_s_at | NHP2 | 55651 | nucleolar protein family A, member 2 |
| | | | | (H/ACA small nucleolar RNPs) |
| 0.62 | 230326_s_at | C11orf73 | 51501 | chromosome 11 open reading frame 73 |
| 0.62 | 221791_s_at | CCDC72 | 51372 | coiled-coil domain containing 72 |
| 0.62 | 201735_s_at | CLCN3 | 1182 | chloride channel 3 |
| 0.62 | 208398_s_at | TBPL1 | 9519 | TBP-like 1 |
| 0.62 | 218200_s_at | NDUFB2 | 4708 | NADH dehydrogenase (ubiquinone) 1 beta |
| | | | | subcomplex, 2, 8 kDa |
| 0.61 | 201381_x_at | CACYBP | 27101 | calcyclin binding protein |
| 0.61 | 224762_at | SERINC2 | 23231 /// | KIAA0746 protein /// serine incorporator 2 |
| | | | 347735 | |
| 0.61 | 215773_x_at | PARP2 | 10038 | poly (ADP-ribose) polymerase 2 |
| 0.61 | 222701_s_at | CHCHD7 | 79145 | coiled-coil-helix-coiled-coil-helix domain |
| | | | | containing 7 |
| 0.61 | 239753_at | LOC44138 | 441383 | hypothetical gene supported by AF086559; |
| | | 3 | | BC065734 |
| 0.6 | 61297_at | CASKIN2 | 57513 | CASK interacting protein 2 |
| 0.6 | 1555764_s_at | TIMM10 | 26519 | translocase of inner mitochondrial membrane |
| | | | | 10 homolog (yeast) |
| 0.59 | 209832_s_at | CDT1 | 81620 | chromatin licensing and DNA replication |
| | | | | factor 1 |
| 0.59 | 226896_at | CHCHD1 | 118487 | coiled-coil-helix-coiled-coil-helix domain |
| | | | | containing 1 |
| 0.59 | 218860_at | NOC4L | 79050 | nucleolar complex associated 4 homolog |
| | | | | (S. cerevisiae) |
| 0.59 | 222027_at | NUCKS1 | 64710 | Nuclear casein kinase and cyclin-dependent |
| | | | | kinase substrate 1 |
| 0.58 | 227941_at | LOC33980 | 339803 | hypothetical protein LOC339803 |
| | | 3 | | |
| 0.58 | 220239_at | KLHL7 | 55975 | kelch-like 7 (Drosophila) |
| 0.58 | 222654_at | IMPAD1 | 54928 | inositol monophosphatase domain containing |
| | | | | 1 |
| 0.58 | 203802_x_at | NSUN5 | 55695 | NOL1/NOP2/Sun domain family, member 5 |
| 0.58 | 212306_at | CLASP2 | 23122 | cytoplasmic linker associated protein 2 |
| 0.58 | 227694_at | C1orf201 | 90529 | chromosome 1 open reading frame 201 |
| 0.58 | 220716_at | GNL3LP | 80060 | guanine nucleotide binding protein-like 3 |
| | | | | (nucleolar)-like pseudogene |
| 0.58 | 1559946_s_at | RUVBL2 | 10856 | RuvB-like 2 (E. coli) |
| 0.57 | 202900_s_at | NUP88 | 4927 | nucleoporin 88 kDa |
| 0.57 | 226845_s_at | MYEOV2 | 150678 | myeloma overexpressed 2 |
| 0.57 | 224947_at | RNF26 | 79102 | ring finger protein 26 |
| 0.57 | 203897_at | LYRM1 | 57149 | LYR motif containing 1 |
| 0.57 | 203867_s_at | NLE1 | 54475 | notchless homolog 1 (Drosophila) |
| 0.57 | 201307_at | 40432 | 55752 | septin 11 |
| 0.57 | 204151_x_at | AKR1C1 | 1645 | aldo-keto reductase family 1, member C1 |
| | | | | (dihydrodiol dehydrogenase 1; 20-alpha |
| | | | | (3-alpha)-hydroxysteroid dehydrogenase) |
| 0.56 | 203606_at | NDUFS6 | 4726 | NADH dehydrogenase (ubiquinone) Fe—S |
| | | | | protein 6, 13 kDa (NADH-coenzyme Q |
| | | | | reductase) |
| 0.56 | 211594_s_at | MRPL9 | 65005 | mitochondrial ribosomal protein L9 |
| 0.56 | 212788_x_at | FTL | 2512 | ferritin, light polypeptide |
| 0.56 | 211162_x_at | SCD | 6319 | stearoyl-CoA desaturase (delta-9-desaturase) |
| 0.56 | 209026_x_at | TUBB | 203068 | tubulin, beta |
| 0.56 | 222979_s_at | SURF4 | 6836 | surfeit 4 |
| 0.55 | 227628_at | GPX8 | 493869 | glutathione peroxidase 8 |
| 0.55 | 204779_s_at | HOXB7 | 3217 | homeobox B7 |
| 0.55 | 224204_x_at | ARNTL2 | 56938 | aryl hydrocarbon receptor nuclear |
| | | | | translocator-like 2 |
| 0.55 | 222653_at | PNPO | 55163 | pyridoxamine 5′-phosphate oxidase |
| 0.55 | 221227_x_at | COQ3 | 51805 | coenzyme Q3 homolog, methyltransferase |
| | | | | (S. cerevisiae) |
| 0.55 | 203967_at | CDC6 | 990 | cell division cycle 6 homolog (S. cerevisiae) |
| 0.55 | 206441_s_at | COMMD4 | 54939 | COMM domain containing 4 |
| 0.55 | 219306_at | KIF15 | 56992 | kinesin family member 15 |
| 0.54 | 201113_at | TUFM | 7284 | Tu translation elongation factor, |
| | | | | mitochondrial |
| 0.54 | 208827_at | PSMB6 | 5694 | proteasome (prosome, macropain) subunit, |
| | | | | beta type, 6 |
| 0.54 | 212380_at | FTSJD2 | 23070 | FtsJ methyltransferase domain containing 2 |
| 0.54 | 226296_s_at | MRPS15 | 64960 | mitochondrial ribosomal protein S15 |
| 0.54 | 226287_at | CCDC34 | 91057 | coiled-coil domain containing 34 |
| 0.54 | 221434_s_at | C14orf156 | 81892 | chromosome 14 open reading frame 156 |
| 0.54 | 224334_s_at | MRPL51 | 10558 /// | mitochondrial ribosomal protein L51 /// |
| | | /// SPTLC1 | 51258 | serine palmitoyltransferase, long chain base |
| | | | | subunit 1 |
| 0.54 | 214264_s_at | C14orf143 | 90141 | chromosome 14 open reading frame 143 |
| 0.53 | 203968_s_at | CDC6 | 990 | cell division cycle 6 homolog (S. cerevisiae) |
| 0.53 | 201577_at | NME1 | 4830 /// | non-metastatic cells 1, protein (NM23A) |
| | | | 4831 | expressed in /// non-metastatic cells 2, |
| | | | | protein (NM23B) expressed in |
| 0.53 | 208447_s_at | PRPS1 | 5631 | phosphoribosyl pyrophosphate synthetase 1 |
| 0.53 | 218580_x_at | AURKAIP | 54998 | aurora kinase A interacting protein 1 |
| | | 1 | | |
| 0.53 | 210125_s_at | BANF1 | 8815 | barrier to autointegration factor 1 |
| 0.53 | 224879_at | C9orf123 | 90871 | chromosome 9 open reading frame 123 |
| 0.53 | 230884_s_at | SPG7 | 6687 | spastic paraplegia 7 (pure and complicated |
| | | | | autosomal recessive) |
| 0.52 | 223759_s_at | GSG2 | 83903 | germ cell associated 2 (haspin) |
| 0.52 | 202839_s_at | NDUFB7 | 4713 | NADH dehydrogenase (ubiquinone) 1 beta |
| | | | | subcomplex, 7, 18 kDa |
| 0.52 | 220459_at | MCM3AP | 114044 | minichromosome maintenance complex |
| | | AS | | component 3 associated protein antisense |
| 0.52 | 224859_at | CD276 | 80381 | CD276 molecule |
| 0.52 | 219288_at | C3orf14 | 57415 | chromosome 3 open reading frame 14 |
| 0.52 | 209714_s_at | CDKN3 | 1033 | cyclin-dependent kinase inhibitor 3 |
| 0.51 | 201797_s_at | VARS | 7407 | valyl-tRNA synthetase |
| 0.51 | 214214_s_at | C1QBP | 708 | complement component 1, q subcomponent |
| | | | | binding protein |
| 0.51 | 219234_x_at | SCRN3 | 79634 | secernin 3 |
| 0.51 | 225614_at | SAAL1 | 113174 | serum amyloid A-like 1 |
| 0.5 | 203105_s_at | DNM1L | 10059 | dynamin 1-like |
| 0.5 | 203744_at | HMGB3 | 3149 | high-mobility group box 3 |
| 0.5 | 201692_at | SIGMAR1 | 10280 | opioid receptor, sigma 1 |
| 0.5 | 205055_at | ITGAE | 3682 | integrin, alpha E (antigen CD103, human |
| | | | | mucosal lymphocyte antigen 1; alpha |
| | | | | polypeptide) |
| 0.5 | 229067_at | SRGAP2P | 653464 | SLIT-ROBO Rho GTPase activating protein |
| | | 1 | | 2 pseudogene 1 |
| 0.5 | 224247_s_at | MRPS10 | 55173 | mitochondrial ribosomal protein S10 |
| 0.5 | 225126_at | MRRF | 92399 | mitochondrial ribosome recycling factor |
| 0.49 | 233539_at | NAPEPLD | 222236 | N-acyl phosphatidylethanolamine |
| | | | | phospholipase D |
| 0.49 | 218100_s_at | IFT57 | 55081 | intraflagellar transport 57 homolog |
| | | | | (Chlamydomonas) |
| 0.49 | 225062_at | LOC38983 | 1001321 | hypothetical protein LOC100132181 /// |
| | | 1 | 81 /// | hypothetical gene supported by AL713796 |
| | | | 389831 | |
| 0.49 | 226936_at | C6orf173 | 387103 | chromosome 6 open reading frame 173 |
| 0.49 | 204036_at | LPAR1 | 1902 | lysophosphatidic acid receptor 1 |
| 0.49 | 218726_at | HJURP | 55355 | Holliday junction recognition protein |
| 0.49 | 239761_at | GCNT1 | 2650 | glucosaminyl (N-acetyl) transferase 1, core 2 |
| | | | | (beta-1,6-N-acetylglucosaminyltransferase) |
| 0.49 | 202415_s_at | HSPBP1 | 23640 | hsp70-interacting protein |
| 0.48 | 202780_at | OXCT1 | 5019 | 3-oxoacid CoA transferase 1 |
| 0.48 | 224209_s_at | GDA | 9615 | guanine deaminase |
| 0.48 | 209836_x_at | BOLA2 /// | 552900 | bolA homolog 2 (E. coli) /// bolA homolog |
| | | BOLA2B | /// | 2B (E. coli) |
| | | | 654483 | |
| 0.48 | 229442_at | C18orf54 | 162681 | chromosome 18 open reading frame 54 |
| 0.48 | 219275_at | PDCD5 | 9141 | programmed cell death 5 |
| 0.48 | 225046_at | LOC38983 | 1001321 | hypothetical protein LOC100132181 |
| | | 1 | 81 | |
| 0.48 | 213187_x_at | FTL | 2512 | ferritin, light polypeptide |
| 0.48 | 235356_at | NHLRC2 | 374354 | NHL repeat containing 2 |
| 0.47 | 225552_x_at | AURKAIP | 54998 | aurora kinase A interacting protein 1 |
| | | 1 | | |
| 0.47 | 1568957_x_at | SRGAP2P | 653464 | SLIT-ROBO Rho GTPase activating protein |
| | | 1 | | 2 pseudogene 1 |
| 0.47 | 200790_at | ODC1 | 4953 | ornithine decarboxylase 1 |
| 0.47 | 222029_x_at | PFDN6 | 10471 | prefoldin subunit 6 |
| 0.47 | 226663_at | ANKRD10 | 55608 | ankyrin repeat domain 10 |
| 0.47 | 222522_x_at | MRPS10 | 55173 | mitochondrial ribosomal protein S10 |
| 0.47 | 225656_at | EFHC1 | 114327 | EF-hand domain (C-terminal) containing 1 |
| 0.47 | 219271_at | GALNT14 | 79623 | UDP-N-acetyl-alpha-D-galactosamine: |
| | | | | polypeptide N-acetylgalactosaminyltransferase 14 |
| | | | | (GalNAc-T14) |
| 0.47 | 215022_x_at | ZNF33B | 7582 | zinc finger protein 33B |
| 0.46 | 213599_at | OIP5 | 11339 | Opa interacting protein 5 |
| 0.46 | 200658_s_at | PHB | 5245 | prohibitin |
| 0.46 | 203428_s_at | ASF1A | 25842 | ASF1 anti-silencing function 1 homolog A |
| | | | | (S. cerevisiae) |
| 0.46 | 227212_s_at | PHF19 | 26147 | PHD finger protein 19 |
| 0.46 | 1555841_at | C9orf30 | 8577 /// | chromosome 9 open reading frame 30 /// |
| | | | 91283 | transmembrane protein with EGF-like and |
| | | | | two follistatin-like domains 1 |
| 0.45 | 203832_at | SNRPF | 6636 | small nuclear ribonucleoprotein polypeptide |
| | | | | F |
| 0.45 | 217553_at | MGC8704 | 256227 | similar to Six transmembrane epithelial |
| | | 2 | | antigen of prostate |
| 0.45 | 203328_x_at | IDE | 3416 | insulin-degrading enzyme |
| 0.45 | 242418_at | C2orf27A | 29798 | Chromosome 2 open reading frame 27 |
| 0.45 | 224753_at | CDCA5 | 113130 | cell division cycle associated 5 |
| 0.44 | 1553978_at | LOC72999 | 1001330 | hypothetical protein LOC100133072 /// |
| | | 1 | 72 /// | hypothetical LOC729991 /// myocyte |
| | | | 4207 /// | enhancer factor 2B |
| | | | 729991 | |
| 0.44 | 219709_x_at | FAM173A | 65990 | family with sequence similarity 173, member |
| | | | | A |
| 0.44 | 226241_s_at | MRPL52 | 122704 | mitochondrial ribosomal protein L52 |
| 0.44 | 202144_s_at | ADSL | 158 | adenylosuccinate lyase |
| 0.44 | 213302_at | PFAS | 5198 | phosphoribosylformylglycinamidine synthase |
| 0.44 | 202870_s_at | CDC20 | 991 | cell division cycle 20 homolog (S. cerevisiae) |
| 0.43 | 209267_s_at | SLC39A8 | 64116 | solute carrier family 39 (zinc transporter), |
| | | | | member 8 |
| 0.43 | 233255_s_at | BIVM | 54841 | basic, immunoglobulin-like variable motif |
| | | | | containing |
| 0.43 | 226537_at | HINT3 | 135114 | histidine triad nucleotide binding protein 3 |
| 0.43 | 220035_at | NUP210 | 23225 | nucleoporin 210 kDa |
| 0.43 | 201272_at | AKR1B1 | 231 | aldo-keto reductase family 1, member B1 |
| | | | | (aldose reductase) |
| 0.42 | 223307_at | CDCA3 | 83461 | cell division cycle associated 3 |
| 0.42 | 213829_x_at | RTEL1 | 51750 | regulator of telomere elongation helicase 1 |
| 0.42 | 219637_at | ARMC9 | 80210 | armadillo repeat containing 9 |
| 0.42 | 222369_at | NAT11 | 79829 | N-acetyltransferase 11 |
| 0.42 | 223435_s_at | PCDHA1 | 56134 /// | protocadherin alpha 1 /// protocadherin alpha |
| | | /// | 56135 /// | 10 /// protocadherin alpha 11 /// |
| | | PCDHA10 | 56136 /// | protocadherin alpha 12 /// protocadherin |
| | | /// | 56137 /// | alpha 13 /// protocadherin alpha 2 /// |
| | | PCDHA11 | 56138 /// | protocadherin alpha 3 /// protocadherin alpha |
| | | /// | 56139 /// | 4 /// protocadherin alpha 5 /// protocadherin |
| | | PCDHA12 | 56140 /// | alpha 6 /// protocadherin alpha 7 /// |
| | | /// | 56141 /// | protocadherin alpha 8 /// protocadherin alpha |
| | | PCDHA13 | 56142 /// | 9 /// protocadherin alpha subfamily C, 1 /// |
| | | /// | 56143 /// | protocadherin alpha subfamily C, 2 |
| | | PCDHA2 | 56144 /// | |
| | | /// | 56145 /// | |
| | | PCDHA3 | 56146 /// | |
| | | /// | 56147 /// | |
| | | PCDHA4 | 9752 | |
| | | /// | | |
| | | PCDHA5 | | |
| | | /// | | |
| | | PCDHA6 | | |
| | | /// | | |
| | | PCDHA7 | | |
| | | /// | | |
| | | PCDHA8 | | |
| | | /// | | |
| | | PCDHA9 | | |
| | | /// | | |
| | | PCDHAC1 | | |
| | | /// | | |
| | | PCDHAC2 | | |
| 0.41 | 211980_at | COL4A1 | 1282 | collagen, type IV, alpha 1 |
| 0.41 | 227295_at | IKIP | 121457 | IKK interacting protein |
| 0.41 | 218980_at | FHOD3 | 80206 | formin homology 2 domain containing 3 |
| 0.4 | 212190_at | SERPINE2 | 5270 | serpin peptidase inhibitor, clade E (nexin, |
| | | | | plasminogen activator inhibitor type 1), |
| | | | | member 2 |
| 0.4 | 236957_at | CDCA2 | 157313 | cell division cycle associated 2 |
| 0.4 | 214960_at | API5 | 8539 | apoptosis inhibitor 5 |
| 0.4 | 232881_at | GNASAS | 149775 | GNAS antisense |
| 0.4 | 224870_at | KIAA0114 | 57291 | KIAA0114 |
| 0.39 | 229070_at | C6orf105 | 84830 | chromosome 6 open reading frame 105 |
| 0.39 | 220840_s_at | C1orf112 | 55732 | chromosome 1 open reading frame 112 |
| 0.39 | 232278_s_at | DEPDC1 | 55635 | DEP domain containing 1 |
| 0.38 | 203114_at | SSSCA1 | 10534 | Sjogren syndrome/scleroderma autoantigen 1 |
| 0.38 | 1552277_a_at | C9orf30 | 8577 /// | chromosome 9 open reading frame 30 /// |
| | | | 91283 | transmembrane protein with EGF-like and |
| | | | | two follistatin-like domains 1 |
| 0.38 | 225967_s_at | C17orf89 | 284184 | chromosome 17 open reading frame 89 |
| 0.37 | 209642_at | BUB1 | 699 | BUB1 budding uninhibited by |
| | | | | benzimidazoles 1 homolog (yeast) |
| 0.37 | 205115_s_at | RBM19 | 9904 | RNA binding motif protein 19 |
| 0.37 | 209263_x_at | TSPAN4 | 7106 | tetraspanin 4 |
| 0.37 | 223253_at | EPDR1 | 54749 | ependymin related protein 1 (zebrafish) |
| 0.37 | 224523_s_at | C3orf26 | 84319 | chromosome 3 open reading frame 26 |
| 0.37 | 219990_at | E2F8 | 79733 | E2F transcription factor 8 |
| 0.37 | 203633_at | CPT1A | 1374 | carnitine palmitoyltransferase 1A (liver) |
| 0.37 | 202580_x_at | FOXM1 | 2305 | forkhead box M1 |
| 0.36 | 237145_at | EIF2AK4 | 440275 | eukaryotic translation initiation factor 2 alpha |
| | | | | kinase 4 |
| 0.36 | 205401_at | AGPS | 8540 | alkylglycerone phosphate synthase |
| 0.36 | 227928_at | C12orf48 | 55010 | chromosome 12 open reading frame 48 |
| 0.36 | 204603_at | EXO1 | 9156 | exonuclease 1 |
| 0.36 | 220060_s_at | C12orf48 | 55010 | chromosome 12 open reading frame 48 |
| 0.36 | 210519_s_at | NQO1 | 1728 | NAD(P)H dehydrogenase, quinone 1 |
| 0.36 | 219926_at | POPDC3 | 64208 | popeye domain containing 3 |
| 0.36 | 225782_at | MSRB3 | 253827 | methionine sulfoxide reductase B3 |
| 0.35 | 205097_at | SLC26A2 | 1836 | solute carrier family 26 (sulfate transporter), |
| | | | | member 2 |
| 0.35 | 204839_at | POP5 | 51367 | processing of precursor 5, ribonuclease |
| | | | | P/MRP subunit (S. cerevisiae) |
| 0.34 | 209891_at | SPC25 | 57405 | SPC25, NDC80 kinetochore complex |
| | | | | component, homolog (S. cerevisiae) |
| 0.34 | 236075_s_at | LOC10012 | 1001296 | similar to hCG2042915 |
| | | 9673 | 73 | |
| 0.34 | 202468_s_at | CTNNAL1 | 8727 | catenin (cadherin-associated protein), |
| | | | | alpha-like 1 |
| 0.34 | 204822_at | TTK | 7272 | TTK protein kinase |
| 0.33 | 209277_at | TFPI2 | 7980 | tissue factor pathway inhibitor 2 |
| 0.33 | 207165_at | HMMR | 3161 | hyaluronan-mediated motility receptor |
| | | | | (RHAMM) |
| 0.33 | 213943_at | TWIST1 | 7291 | twist homolog 1 (Drosophila) |
| 0.33 | 209278_s_at | TFPI2 | 7980 | tissue factor pathway inhibitor 2 |
| 0.32 | 235572_at | SPC24 | 147841 | SPC24, NDC80 kinetochore complex |
| | | | | component, homolog (S. cerevisiae) |
| 0.31 | 206343_s_at | NRG1 | 3084 | neuregulin 1 |
| 0.31 | 227896_at | BCCIP | 56647 | BRCA2 and CDKN1A interacting protein |
| 0.3 | 205376_at | INPP4B | 8821 | inositol polyphosphate-4-phosphatase, type |
| | | | | II, 105 kDa |
| 0.3 | 214240_at | GAL | 51083 | galanin prepropeptide |
| 0.3 | 229362_at | PUS10 | 150962 | Pseudouridylate synthase 10 |
| 0.3 | 203162_s_at | KATNB1 | 10300 | katanin p80 (WD repeat containing) subunit |
| | | | | B1 |
| 0.29 | 230508_at | DKK3 | 27122 | dickkopf homolog 3 (Xenopus laevis) |
| 0.29 | 201467_s_at | NQ01 | 1728 | NAD(P)H dehydrogenase, quinone 1 |
| 0.27 | 207517_at | LAMC2 | 3918 | laminin, gamma 2 |
| 0.27 | 223404_s_at | C1orf25 | 81627 | chromosome 1 open reading frame 25 |
| 0.26 | 223700_at | MND1 | 84057 | meiotic nuclear divisions 1 homolog |
| | | | | (S. cerevisiae) |
| 0.26 | 204619_s_at | VCAN | 1462 | versican |
| 0.25 | 226611_s_at | CENPV | 201161 | proline rich 6 |
| 0.25 | 213043_s_at | MED24 | 9862 | mediator complex subunit 24 |
| 0.25 | 1558683_a_at | HMGA2 | 8091 | high mobility group AT-hook 2 |
| 0.24 | 225834_at | FAM72A | 653573 | family with sequence similarity 72, member |
| | | /// | /// | A /// family with sequence similarity 72, |
| | | FAM72B | 653820 | member B /// gastric cancer up-regulated-2 |
| | | /// | /// | |
| | | FAM72C | 729533 | |
| | | /// | | |
| | | FAM72D | | |
| 0.22 | 229778_at | C12orf39 | 80763 | chromosome 12 open reading frame 39 |
| 0.19 | 202275_at | G6PD | 2539 | glucose-6-phosphate dehydrogenase |
| 0.16 | 1555225_at | C1orf43 | 25912 | chromosome 1 open reading frame 43 |
| 0.12 | 244623_at | KCNQ5 | 56479 | potassium voltage-gated channel, KQT-like |
| | | | | subfamily, member 5 |
| 0.12 | 1558152_at | LOC10013 | 1001312 | hypothetical protein LOC100131262 |
| | | 1262 | 62 | |
| 0.11 | 1561633_at | HMGA2 | 8091 | high mobility group AT-hook 2 |
| 0.09 | 210143_at | ANXA10 | 11199 | annexin A10 |

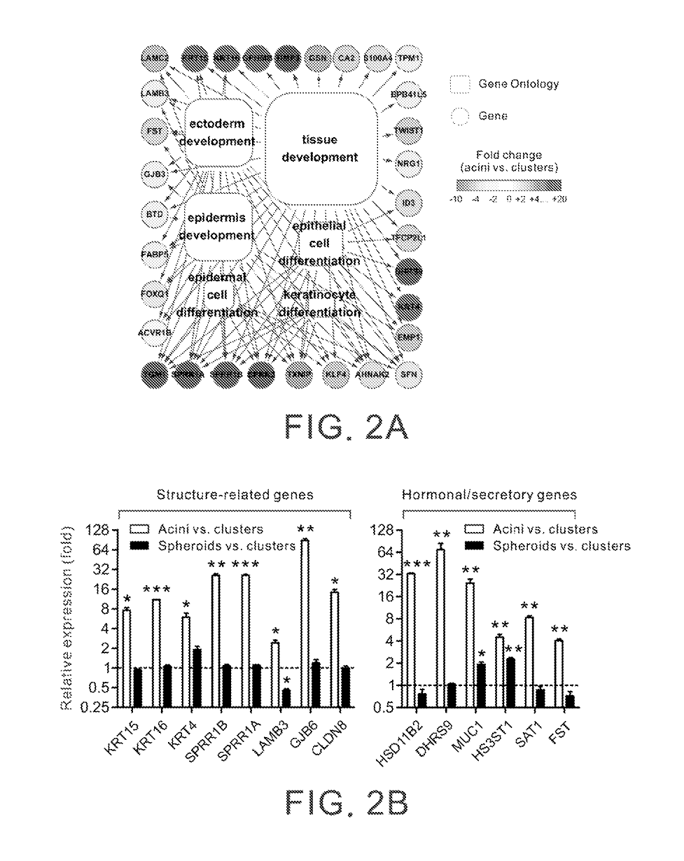

In FIGS. 2A and 2B, Gene Ontology functional clustering analysis revealed that the genes in this set of 411 genes that were up-regulated during prostatic acinar differentiation were substantially enriched for those related to epithelial and ectodermal differentiation and maintenance of epithelial architectures (FIG. 2A), including the cytokeratin proteins KRT15, KRT16 and KRT4, the keratinocyte membranous proteins, SPRR1B and SPRR1A, the laminin-5 subunits LAMB3, the gap junction protein GJB6 and GJB3, the tight junction protein CLDN8, and the differentiation-associated transcriptional factors KLF4 and FOXQ1, as well as factors related to the hormonal and secretory functions of prostatic glands, including steroid and progesterone metabolism (HSD11B2, DHRS9), mucin or heparin sulfate production (MUC1, HS3ST1), spermidine/spermine metabolism (SAT1), and the gonadal protein (FST) (FIG. 2B). These findings lend strong supports to our tissue organization model as a valid way to capture the molecular signals specific to the structural and functional differentiation processes of prostatic glands.

### Example 2

This example demonstrates that prostate cancers carrying the expression profile of the 411-gene in differentiated prostatic acini link to favorable clinical prognosis.

To demonstrate if the molecular profile associated with prostatic acinar differentiation carries important prognostic information in human prostate cancer, we interrogated a published gene expression microarray data set consisting of 21 patients with localized prostate cancer who underwent radical prostatectomy at the Brigham and Woman's Hospital (Boston, Mass.; the BWH cohort) (Singh et al., 2002). We determined the degree of resemblance between the patient tumors and prostatic acini by calculating the Pearson's correlation coefficients (r_{acini}) based on the expression of the 411 acinar differentiation-related genes.

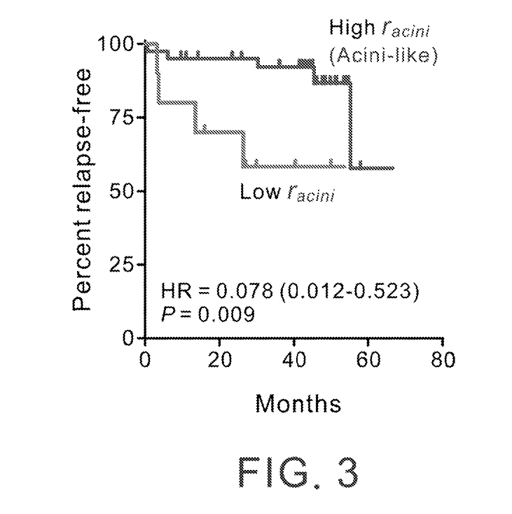

In FIG. 3, the patients were divided into two subgroups according to r_{acini}, with the threshold determined by the maximal Youden's index (Pepe, 2003). We designated the tumors with higher r_{acini} “acini-like” tumors and found that patients with this type of tumors exhibited significantly lower risk for relapse compared to those with lower correlation values by Kaplan-Meier analysis (log-rank test P=0.009). The estimated 3-year rate of relapse-free survival was 92.1% among patients with acini-like PCA, and 58.3% in those in the group with lower r_{acini}.

As shown in Table 2, in a multivariate Cox proportional-hazards analysis, the r_{acini} of the tumors was found to be the only significant predictor of relapse (hazard ratio=0.173 (0.041-0.725), P=0.016).

TABLE 2
Multivariate Cox regression model predicting recurrence by racini
and clinical and pathological criteria in the BWH cohort.

| | | 95% Confidence | |
|---|---|---|---|
| | Hazard ratio | Interval | P-value |
| Patient age (years) | 0.997 | 0.888-1.118 | 0.956 |
| Tumor stage (stage 3 vs. | 1.085 | 0.242-4.863 | 0.915 |
| stage 2) | | | |
| Serum prostate-specific | 1.002 | 0.856-1.172 | 0.981 |
| antigen | | | |
| Gleason score (>=7 vs. <6) | 2.182 | 0.420-11.334 | 0.354 |
| r_{acini} (high vs. low) | 0.173 | 0.041-0.725 | 0.016 |

To assess how robustly the expression profile of prostatic acini can stratify risk of relapse in prostate cancer, we repeated the above analysis in an independent tumor transcriptome data set derived from 29 prostate cancer patients who had received radical prostatectomy and had been followed up for up to 5 years (Lapointe et al., 2004).

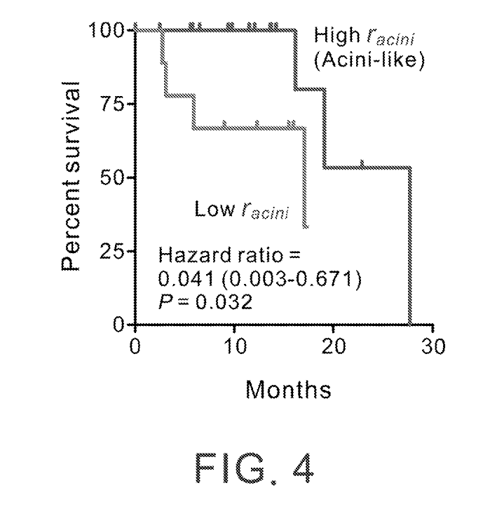

FIG. 4 shows that the patient with higher r_{acini} (i.e., acini-like tumors) fared better than those with lower r_{acini} in this validation set (log-rank test P=0.032), with an estimated 18-month relapse-free survival of 80% among patients in the group with a higher r_{acini} and 0% in those in the group with a lower correlation values.

As shown in Table 3, multivariate Cox regression analysis confirmed that r_{acini} provided independent prognostic information in prostate cancer while the Gleason score was only marginally prognostic in this cohort.

TABLE 3
Multivariate Cox regression model predicting recurrence by r_{acini} and
clinical and pathological criteria in the Lapointe et al. cohort)

| | | 95% Confidence | |
|---|---|---|---|
| | Hazard ratio | Interval | P-value |
| Patient age (years) | 0.969 | 0.743-1.264 | 0.816 |
| Tumor stage (stage 3 vs. | 11.103 | 0.867-142.232 | 0.064 |
| stage 2) | | | |
| Gleason score (>=7 vs. <6) | 4.398 | 0.452-42.761 | 0.202 |
| r_{acini} (high vs. low) | 0.041 | 0.003-0.671 | 0.025 |

### Example 3

This example describes the identification of a 12-gene prognostic model of prostate cancer based on the molecular profile related to prostatic acinar differentiation.

Having demonstrated the prognostic value of the prostatic acini-related expression profile in prostate cancer, we sought to refine this profile and identify a smaller set of genes with higher clinical utility. To this end, we mapped the 411 acini-related genes to the BWH data set (Singh et al., 2002) and constructed a “recurrence score” based on a Cox's model to predict the occurrence of tumor relapse following radical prostatectomy. We used a previously described supervised approach with modifications (Wang et al., 2005). Briefly, for each gene, univariate Cox's regression analysis was used to measure the correlation between the expression level of the gene (on a log₂ scale) and the length of relapse-free survival of the PCA patients in the BWH cohort. We constructed 1000 bootstrap samples of the patients in the cohort and performed Cox's regression analysis on each of the samples. We then determined an estimated P-value and an estimated standardized Cox regression coefficient for each gene by calculating the median P-values and the median Cox's coefficient of the 1000 bootstrap samples, respectively. To ensure the consistency of our model, we selected the genes whose expressional changes during prostatic acinar differentiation were associated with the expected positive (for genes up-regulated in cell clusters) or negative risk of relapse (for genes up-regulated in prostatic acini), as determined by the estimated standardized Cox regression coefficient. The selected genes were then ranked-ordered according to the estimated P-values, and multiple sets of genes were generated by repeatedly adding one more genes each time from top of the descendingly ranked list, starting from the first three top-ranked genes. Then a “recurrence score” (Equation 1) were calculated to measure the risk of post-operative recurrence of a patient for a gene set:

Recurrence score=Σᵢ₌₃^{k}bᵢxᵢ (Equation 1)

where k is the number of probes in the probe set, bᵢ is the standardized Cox regression coefficient for the ith probe and xᵢ is the log₂ expression level for the ith probe.

For each selected probe set the concordance index (C-index) was used to evaluate the predictive accuracy in survival analysis (Pencina and D'Agostino, 2004). C-index statistics analysis was conducted using the ‘survcomp’ package in the statistical programming language R (cran.r-project.org). The gene set that achieved the maximal predictive accuracy while contained the fewest number of the genes was selected as the optimized prognostic predictor.

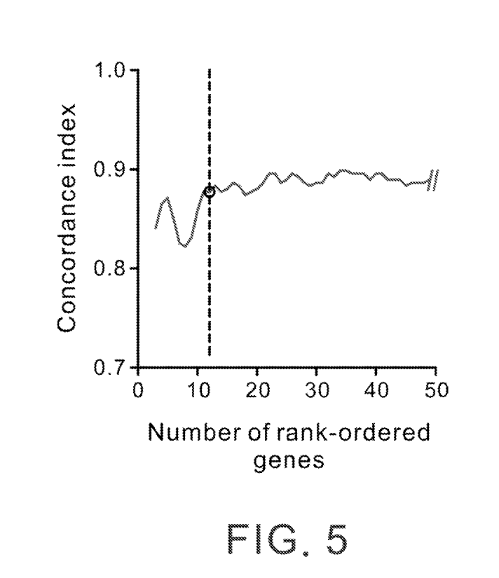

As shown in FIG. 5, through this approach, we selected a set of 12 genes whose performance in the prognostic prediction, as assessed by C-index, reached a plateau.

Table 4 shows the identities of the 12 selected genes.

TABLE 4
Description of genes in the 12-gene signature

| Higher | Hazard by | | | |
|---|---|---|---|---|
| expres- | Cox | | Entrez | |
| sion in | regression | Symbol | gene ID | Gene title |
| Acini | 0.0052 | ST6GALNAC2 | 10610 | ST6 (alpha-N-acetyl- |
| | | | | neuraminyl-2,3-beta- |
| | | | | galactosyl-1,3)-N- |
| | | | | acetylgalactosaminide |
| | | | | alpha-2,6- |
| | | | | sialyltransferase 2 |
| Acini | 0.0041 | ABCG1 | 9619 | ATP-binding cassette, |
| | | | | sub-family G, member 1 |
| Acini | 0.0003 | BTD | 686 | Biotinidase |
| Acini | 0.0071 | PDCD4 | 27250 | Programmed cell death 4 |
| Clusters | 103.5751 | BANF1 | 8815 | Barrier to autointegration |
| | | | | factor 1 |
| Acini | 0.0092 | KLF6 | 1316 | Kruppel-like factor 6 |
| Acini | 0.0471 | IRS1 | 3667 | Insulin receptor substrate |
| | | | | 1 |
| Acini | 0.0146 | ZNF185 | 7739 | Zinc finger protein 185 |
| Acini | 0.0838 | ANXA11 | 311 | Annexin A11 |
| Acini | 0.0088 | DUSP2 | 1844 | Dual specificity |
| | | | | phosphatase 2 |
| Acini | 0.0231 | KLF4 | 9314 | Kruppel-like factor 4 |
| Acini | 0.0199 | DSC2 | 1824 | Desmocollin 2 |

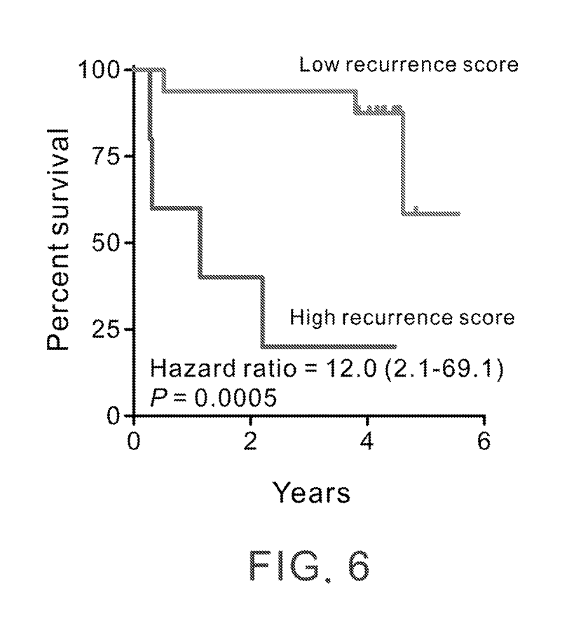

FIG. 6 shows that, based on the recurrence score (Equation 1), the expression profile of this 12 gene signature could very effectively stratify risk of disease recurrence by Kaplan-Meier analysis in the BWH cohort (log-rank test P=0.0005).

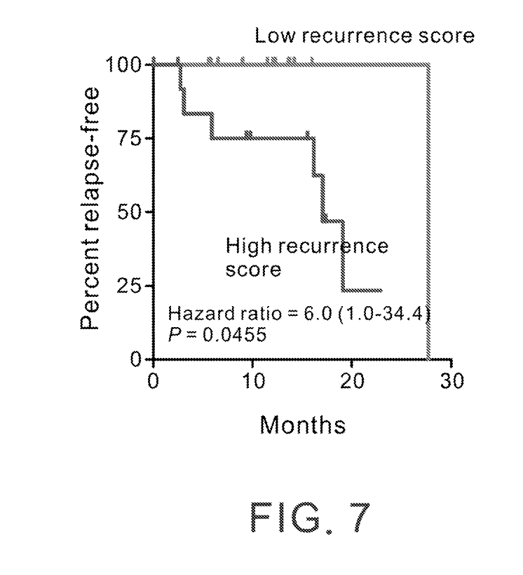

FIG. 7 shows that the recurrence score calculated based on the 12 gene model also stratified the patients in the Lapointe et al. cohort into two groups that exhibited considerable difference in risk for recurrence (log-rank test P=0.0455).

As shown in Table 5, multivariate Cox regression analysis demonstrates that this 12-gene model provides strong and independent prognostic information to prostate cancer (hazard ratio=42.304, P=0.004).

TABLE 5
Multivariate Cox regression model predicting recurrence by the 12-
gene model and clinico-pathological criteria in the BWH cohort.

| | | 95% Confidence | |
|---|---|---|---|
| | Hazard ratio | Interval | P-value |
| Patient age (years) | 1.006 | 0.910-1.111 | 0.910 |
| Tumor stage (3 vs. 2) | 0.938 | 0.211-4.175 | 0.930 |
| Serum PSA | 1.115 | 0.927-1.343 | 0.250 |
| Gleason score (≧7 vs. <6) | 5.255 | 0.633-43.650 | 0.120 |
| Recurrence score (12-gene | 42.304 | 3.323-537.971 | 0.004 |
| model, high vs. low) | | | |

Table 6 shows that the 12-gene model markedly enhanced the prognostic accuracy of a combined clinical model including clinical and pathological variables (C-index from 0.620 to 0.847) and outperformed several previously reported prognostic gene signatures of prostate cancer (Glinsky et al., 2004; Singh et al., 2002).

TABLE 6
The prediction accuracy, as evaluated by the C-index, of
different prognosis prediction models in the BWH cohort.

| | | 95% Confidence | |
|---|---|---|---|
| | C-index | Interval | P-value |
| Combined clinical | 0.620 | 0.418-0.821 | 0.122 |
| model (age, tumor | | | |
| stage, serum PSA, | | | |
| and Gleason score) | | | |
| 5-gene signature | 0.764 | 0.530-0.997 | 0.013 |
| (Singh et al., 2002)* | | | |
| 5-gene signature | 0.767 | 0.562-0.972 | 0.005 |
| (Glinsky et al., 2004)^{†} | | | |
| r_{acini} | 0.777 | 0.543-1.000 | 0.010 |
| 12-gene signature | 0.847 | 0.746-0.947 | <0.001 |

*The 5-gene signature includes chromogranin A (CHGA), platelet-derived growth factor receptor β (PDGFRB), homeobox C6 (HOXC6), inositol triphosphate receptor 3 (IPTR3) and sialyltransferase-1 (ST3GAL1).
^{†}The 5-gene signature includes non-imprinted in Prader-Willi/Angelman syndrome region protein 2 (NIPA2) or HGC5466, wingless-type MMTV integration site family, member 5A (WNT5A), DENN/MADD domain containing 4B (DENND4B) or KIAA0476, inositol 1,4,5-trisphosphate receptor type 1 (ITPR1) and transcription factor 2 (TCF2).

### Example 4

This example describes the prognostic value of the respective markers in Table 4.

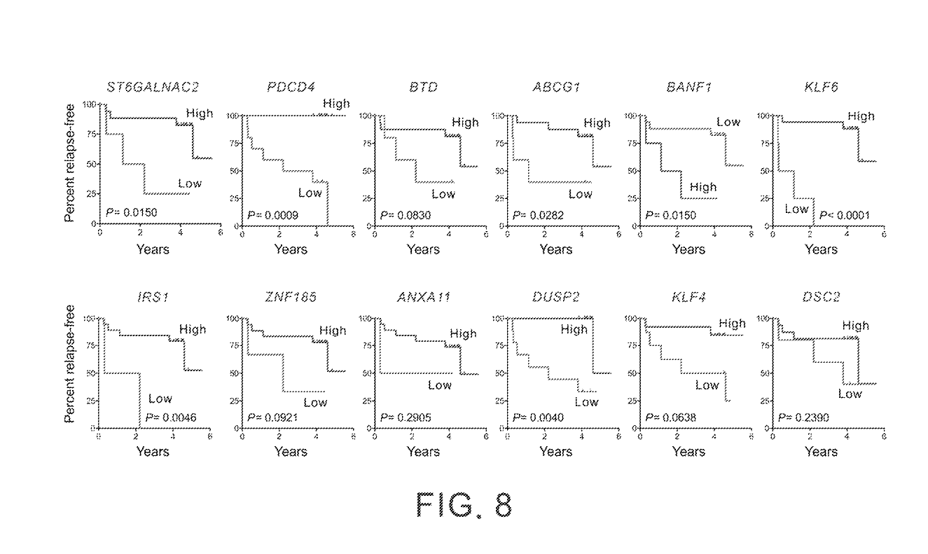

FIG. 8 shows that most of the 12 molecular markers in Table 4 could individually stratify prostate cancer patients in the BWH cohort into two groups that exhibited significant difference in risk for recurrence following radical prostatectomy. The exceptions to this were ANXA11 and DSC2, which were marginally prognostic (log rank test P>0.1). Except BANF1, all of these markers were up-regulated in prostatic acini relative to cell clusters (Table 4) and were associated with lower risks of disease relapse, suggesting their potential roles as markers of tissue differentiation and tumor suppressors. By contrast, the transcript abundance level of BANF1 was down-regulated in prostatic acini and was positively associated with risk of recurrence.

Cancer biomarkers are more clinically applicable if they can be incorporated in routine pathological examinations. To determine if the prognostic correlation of the genes in the 12-gene model could be observed at the protein and the tissue levels in human prostate cancer materials, the tissue expressions of three selected markers, including PDCD4, ABCG1 and KLF6, by performing immunohistochemistry staining of the tumor tissues from an independent cohort of 61 early-stage prostate cancer patients who underwent radical prostatectomy and had been followed up for up to 11 years at Chimei Foundational Medical Center (Tainan, Taiwan; the CFMC cohort). These markers were selected as specific and pathology validated antibodies are commercially available, which included anti-ABCG1 (clone EP1366Y), anti-PDCD4 (clone EPR3431), and anti-KLF6 (all from Epitomics, Burlingame, Calif.). Briefly, formalin-fixed, paraffin-embedded tissues of human prostate cancer and the associated clinical data from 61 patients who received radical prostatectomy at Chimei Foundational Medical Center were acquired and used in conformity with Institutional Review Board-approved protocols (the CFMC cohort). Biochemical recurrence of PCA was defined as a prostate-specific antigen (PSA) of at least 0.4 ng/ml or two consecutive PSA values of 0.2 mg/ml and rising (Stephenson et al., 2006). Tissue sections were deparaffinized, hydrated, immersed in citrate buffer at pH 6.0 for epitope retrieval in a microwave. Endogenous peroxidase activity was quenched in 3% hydrogen peroxidase for 15 minutes, and slides were then incubated with 10% normal horse serum to block nonspecific immunoreactivity. The antibody was subsequently applied and detected by using the DAKO EnVision kit (DAKO). All the immunohistochemical (IHC) staining was evaluated by the same expert pathologist and the staining patterns were quantified using the histological score (H-score) (Budwit-Novotny et al., 1986).

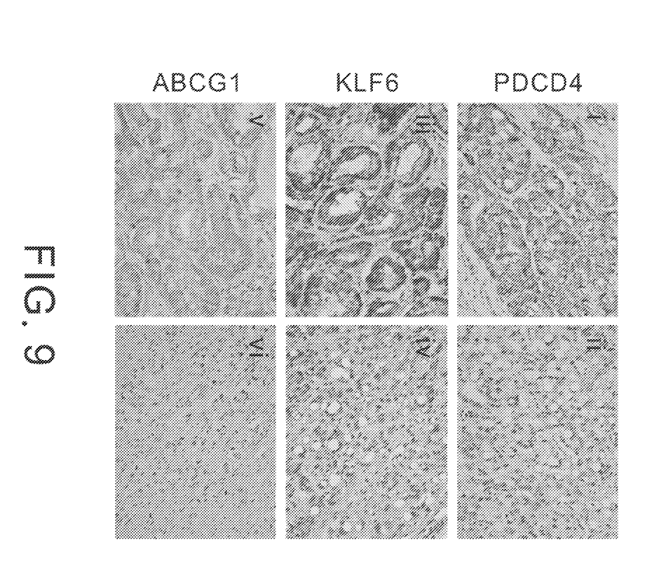

FIG. 9 shows representative immunostaining of PDCD4 (i, ii), KLF6 (iii, iv) and ABCG1 (v, vi) in PCA tissues (400× magnification). The antibodies used include anti-ABCG1 (clone EP1366Y), anti-PDCD4 (clone EPR3431), and anti-KLF6 (all from Epitomics, Burlingame, Calif.). Shown are tumors with high (i, iii, v) or low (ii, iv, vi) staining intensities of the respective markers.

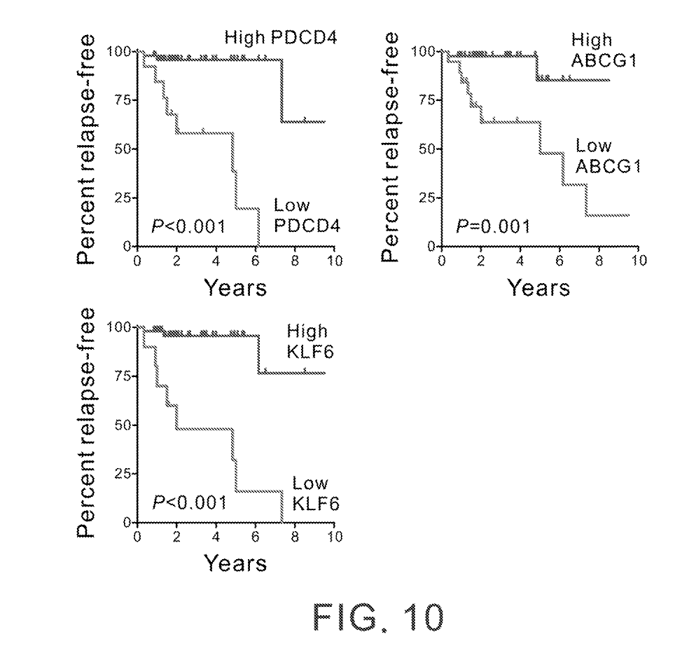

As shown in FIG. 10, the staining intensities of PDCD4, as assessed by the H-score, showed strong negative associations with risk of post-operative biochemical recurrence by Kaplan-Meier analysis (log-rank test P<0.001). Similarly, we found that tumors stained intensely with KLF6 or ABCG1 were associated with significantly longer recurrence-free survival compared to those with lower staining intensities (log-rank test P<0.001, respectively).

As shown in Table 7, multivariate Cox-regression analyses demonstrated that PCDC4, ABCG1 or KLF6 was strongly prognostic independent of clinical criteria and Gleason's score.

TABLE 7
Multivariate Cox regression model predicting recurrence
by the staining intensities of PDCD4, KLF6 or ABCG1 and
clinico-pathological criteria in the CFMC cohort.

| | | |
|---|---|---|
| | 95% Confidence | |
| Hazard ratio | Interval | P-value |

Marker: PDCD4

| | | | |
|---|---|---|---|
| Patient age (years) | 1.004 | 0.847-1.191 | 0.961 |
| Tumor stage (3 vs. <3) | 1.639 | 0.344-7.819 | 0.535 |
| Gleason score (≧7 vs. <6) | 2.314 | 1.125-4.759 | 0.023 |
| Staining intensity | 0.114 | 0.022-0.606 | 0.011 |
| (high vs. low) | | | |

Marker: KLF6

| | | | |
|---|---|---|---|
| Patient age (years) | 0.986 | 0.843-1.153 | 0.861 |
| Tumor stage (3 vs. <3) | 3.106 | 0.676-14.27 | 0.145 |
| Gleason score (≧7 vs. <6) | 1.974 | 0.934-4.176 | 0.075 |
| Staining intensity | 0.164 | 0.039-0.695 | 0.014 |
| (high vs. low) | | | |

Marker: ABCG1

| | | | |
|---|---|---|---|
| Patient age (years) | 0.976 | 0.833-1.142 | 0.758 |
| Tumor stage (3 vs. <3) | 3.079 | 0.644-14.715 | 0.159 |
| Gleason score (≧7 vs. <6) | 2.424 | 1.177-4.99 | 0.016 |
| Staining intensity | 0.187 | 0.036-0.957 | 0.044 |
| (high vs. low) | | | |

### Example 5

This example describes a three-gene prognostic model of prostate cancer based on the expression levels of PDCD4, ABCG1 and KLF6.

In Example 4, three of the gene markers in the 12-gene model of prostate cancer, including PDCD4, ABCG1 and KLF6, can be examined by immunohistochemical staining of prostate tumor tissues. The staining intensities of each of these markers showed strong negative associations with risk of post-operative biochemical recurrence (FIG. 10). Likewise, the mRNA expression levels of PDCD4, ABCG1 or KLF6 showed strong negative associations with risk of post-operative disease relapse (FIG. 8). We therefore assessed whether we could use the expression levels of PDCD4, ABCG1 and KLF6 to establish a three-gene prognostic model of prostate cancer. To this end, we calculated the recurrence score (Equation 1) based on the staining intensities, as quantified by H-score, of PDCD4, ABCG1 and KLF6 in the CFMC cohort. The patients were stratified into two subgroups with high- or low-risk of post-operative biochemical relapse according to the recurrence score with the threshold determined by the maximal Youden's index (Pepe, 2003).

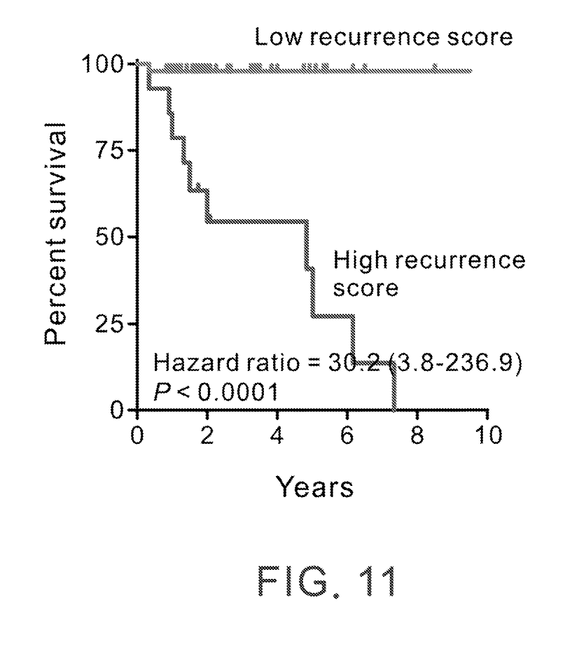

As shown in FIG. 11, based on the recurrence score, the staining intensities of PDCD4, ABCG1 and KLF6 could very effectively stratify risk of disease recurrence by Kaplan-Meier analysis in the CFMC cohort (hazard ratio=30.2, log-rank test P<0.0001). Remarkably, none of the patients in the low risk group developed disease recurrence within the entire follow-up period. By contrast, the medium survival of the patients in the high risk group was 4.833 months.

As shown in Table 8, multivariate Cox regression analysis demonstrates that this three-gene model provides the strongest prognostic information to prostate cancer independent of clinical criteria and Gleason score (hazard ratio=22.591, P=0.004).

TABLE 8
Multivariate Cox regression model predicting recurrence by the three-
gene model and clinico-pathological criteria in the CFMC cohort.

| | | 95% Confidence | |
|---|---|---|---|
| | Hazard ratio | Interval | P-value |
| Patient age (years) | 1.009 | 0.856-1.188 | 0.919 |
| Tumor stage (3 vs. 2) | 3.841 | 0.575-25.654 | 0.165 |
| Serum PSA | 0.984 | 0.948-1.022 | 0.417 |
| Gleason score (≧7 vs. <6) | 8.261 | 0.474-143.880 | 0.148 |
| Recurrence score (3-gene | 22.591 | 2.712-188.158 | 0.004 |
| model, high vs. low) | | | |

Table 9 shows that, according to concordance index (C-index) values (Pencina and D'Agostino, 2004), the predictive accuracy of the three-gene model reached 0.951, which significantly (P=0.001) outperformed a combined clinical model including age, tumor stage, serum PSA, and Gleason score, which had a prediction accuracy of 0.695 by C-statistics.

TABLE 9
The prediction accuracy, as evaluated by the C-index, of the three-
gene model and clinico-pathological criteria in the CFMC cohort.

| | | | P-value | P-value vs. |
|---|---|---|---|---|
| | Concor- | 95% | for | combined |
| | dance | Confidence | C-index | clinical |
| | index | Interval | (vs. 0.5) | model |
| Combined clinical | 0.695 | 0.537-0.854 | 0.0079 | |
| model (age, tumor | | | | |
| stage, serum PSA, | | | | |
| and Gleason score) | | | | |
| Three-gene model | 0.951 | 0.859-1.000 | <0.0001 | 0.001 |
| (PDCD4, ABCG1 | | | | |
| and KLF6) | | | | |

Having demonstrated the outstanding performance of the three-gene prognostic model of prostate cancer, we next tested its performance in the BWH cohort. In this data set, we used the transcript abundance levels of PDCD4, ABCG1 and KLF6 to calculate the recurrence score, and stratified the patients into two subgroups with high- or low-risk of post-operative relapse with the threshold determined by the maximal Youden's index.

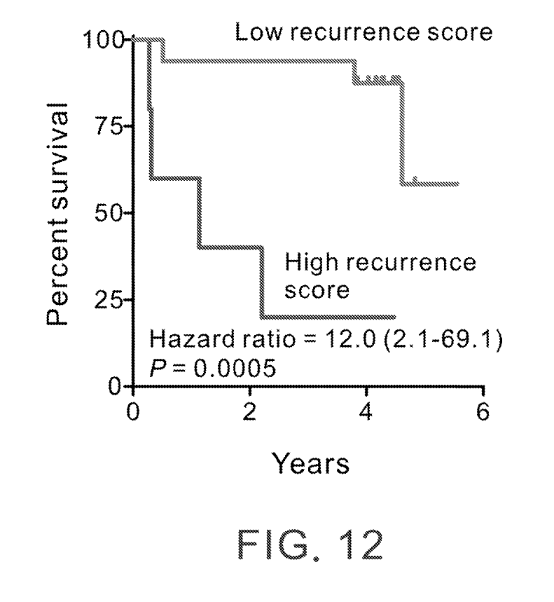

FIG. 12 shows, based on the recurrence score (Equation 1), the transcript abundance levels of PDCD4, ABCG1 and KLF6 could very effectively stratify risk of disease recurrence by Kaplan-Meier analysis in the BWH cohort (hazard ratio=12.0, log-rank test P=0.0005).

As shown in Table 10, multivariate Cox regression analysis demonstrates that this three-gene model provides the strongest and independent prognostic information to prostate cancer with a hazard ratio for post-operative disease relapse reaching 59.551 (P=0.006).

TABLE 10
Multivariate Cox regression model predicting recurrence by the three-
gene model and clinico-pathological criteria in the BWH cohort.

| | | 95% Confidence | |
|---|---|---|---|
| | Hazard ratio | Interval | P-value |
| Patient age (years) | 0.938 | 0.794-1.107 | 0.448 |
| Tumor stage (3 vs. 2) | 0.076 | 0.005-1.094 | 0.058 |
| Serum PSA | 1.316 | 1.007-1.721 | 0.044 |
| Gleason score (≧7 vs. <6) | 2.646 | 0.301-23.278 | 0.381 |
| Recurrence score (3-gene | 59.551 | 3.280-1081-218 | 0.006 |
| model, high vs. low) | | | |

Table 11 shows that, according to C-index, the predictive accuracy of the three-gene model in the BWH cohort reached 0.939 (P<0.001), which markedly (P=0.002) enhanced the prognostic accuracy of a combined clinical model including age, tumor stage, serum PSA, and Gleason score, which by itself did not have significant prognostic value (C-index=0.617, P=0.113).

TABLE 11
The prediction accuracy, as evaluated by the C-index, of the three-
gene model and clinico-pathological criteria in the BWH cohort.

| | | | P-value | P-value vs. |
|---|---|---|---|---|
| | Concor- | 95% | for | combined |
| | dance | Confidence | C-index | clinical |
| | index | Interval | (vs. 0.5) | model |
| Combined clinical | 0.617 | 0.428-0.806 | 0.113 | |
| model (age, tumor | | | | |
| stage, serum PSA, | | | | |
| and Gleason score) | | | | |
| Three-gene model | 0.939 | 0.862-1.000 | <0.001 | 0.002 |
| (PDCD4, ABCG1 | | | | |
| and KLF6) | | | | |

### Example 6

This example describes a two-gene prognostic model of prostate cancer based on the expression levels of PDCD4 and ABCG1.

It was demonstrated that the expression levels of PDCD4 and ABCG1 could be used to establish an effective two-gene prognostic model of prostate cancer. We calculated the recurrence score (Equation 1) based on the staining intensities, as quantified by H-score, of PDCD4 and ABCG1 in the CFMC cohort. The patients were stratified into two subgroups with high- or low-risk of post-operative biochemical relapse according to the recurrence score with the threshold determined by the maximal Youden's index.

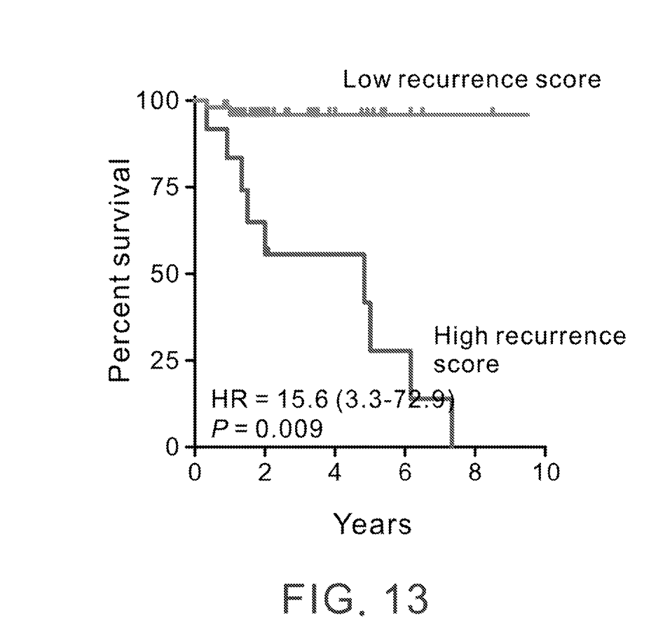

As shown in FIG. 13, based on the recurrence score, the staining intensities of PDCD4 and ABCG1 could very effectively stratify risk of disease recurrence by Kaplan-Meier analysis in the CFMC cohort (hazard ratio=15.6, log-rank test P=0.009).

As shown in Table 12, multivariate Cox regression analysis demonstrates that this two-gene model provides the strongest prognostic information to prostate cancer independent of clinical criteria and Gleason score (hazard ratio=16.25, P=0.002).

TABLE 12
Multivariate Cox regression model predicting recurrence by the two-
gene model and clinico-pathological criteria in the CFMC cohort.

| | | | P-value | P-value vs. |
|---|---|---|---|---|
| | Concor- | 95% | for | combined |
| | dance | Confidence | C-index | clinical |
| | index | Interval | (vs. 0.5) | model |
| Combined clinical | 0.695 | 0.537-0.854 | 0.0079 | |
| model (age, tumor | | | | |
| stage, serum PSA, | | | | |
| and Gleason score) | | | | |
| Two-gene model | 0.915 | 0.801-1.000 | <0.0001 | 0.012 |
| (PDCD4 and ABCG1) | | | | |

Table 13 shows that, according to C-index values, the predictive accuracy of the two-gene model reached 0.915, which significantly (P=0.012) outperformed a combined clinical model including age, tumor stage, serum PSA, and Gleason score.

TABLE 13
The prediction accuracy, as evaluated by C-index, of the two-gene
model and clinico-pathological criteria in the CFMC cohort.

| | | | P-value | P-value vs. |
|---|---|---|---|---|
| | Concor- | 95% | for | combined |
| | dance | Confidence | C-index | clinical |
| | index | Interval | (vs. 0.5) | model |
| Combined clinical | 0.695 | 0.537-0.854 | 0.0079 | |
| model (age, tumor | | | | |
| stage, serum PSA, | | | | |
| and Gleason score) | | | | |
| Two-gene model | 0.915 | 0.801-1.000 | <0.0001 | 0.012 |
| (PDCD4 and ABCG1) | | | | |

The performance of the two-gene prognostic model in the 21-patient BWH cohort was tested next. In this data set, we used the transcript abundance levels of PDCD4 and ABCG1 to calculate the recurrence score, and stratified the patients into two subgroups with high- or low-risk of post-operative relapse.

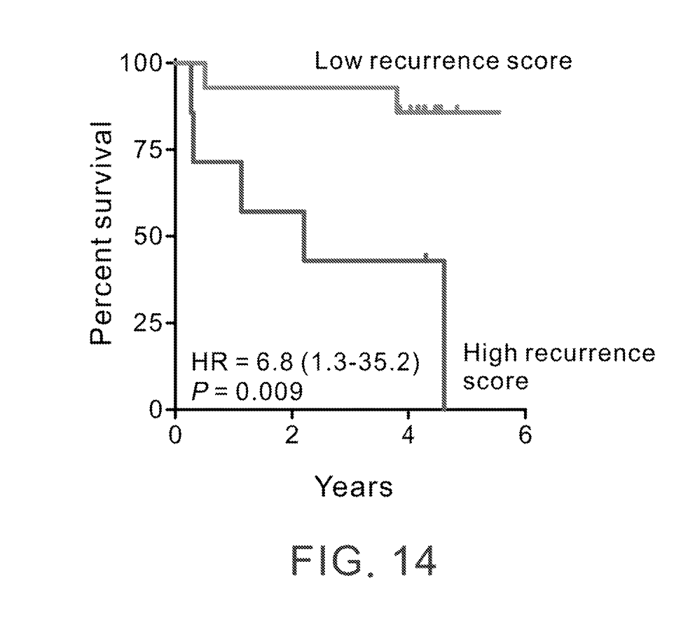

FIG. 14 shows, based on the recurrence score, the transcript abundance levels of PDCD4 and ABCG1 could very effectively stratify risk of disease recurrence by Kaplan-Meier analysis in the BWH cohort (hazard ratio=6.8, log-rank test P=0.009).

As shown in Table 15, multivariate Cox regression analysis demonstrates that this two-gene model provides the strongest and independent prognostic information to prostate cancer with a hazard ratio for post-operative disease relapse reaching 139.963 (P=0.048).

TABLE 14
Multivariate Cox regression model predicting recurrence by the two-
gene model and clinico-pathological criteria in the BWH cohort.

| | | 95% Confidence | |
|---|---|---|---|
| | Hazard ratio | Interval | P-value |
| Patient age (years) | 1.089 | 0.907-1.307 | 0.36 |
| Tumor stage (3 vs. 2) | 0.058 | 0.002-2.165 | 0.124 |
| Serum PSA | 1.478 | 0.944-2.313 | 0.087 |
| Gleason score (≧7 vs. <6) | 15.773 | 0.599-415.027 | 0.098 |
| Recurrence score (2-gene | 139.963 | 1.034-18940-682 | 0.048 |
| model, high vs. low) | | | |

Table 15 shows that, according to C-index, the predictive accuracy of the two-gene model in the BWH cohort reached 0.875 (P<0.001), which significantly (P=0.022) enhanced the prognostic accuracy of a combined clinical model including age, tumor stage, serum PSA, and Gleason score.

TABLE 15
The prediction accuracy, as evaluated by C-index, of the two-gene
model and clinico-pathological criteria in the BWH cohort.

| | | | P-value | P-value vs. |
|---|---|---|---|---|
| | Concor- | 95% | for | combined |
| | dance | Confidence | C-index | clinical |
| | index | Interval | (vs. 0.5) | model |
| Combined clinical | 0.617 | 0.428-0.806 | 0.113 | |
| model (age, tumor | | | | |
| stage, serum PSA, | | | | |
| and Gleason score) | | | | |
| Two-gene model | 0.875 | 0.713-1.000 | <0.001 | 0.022 |
| (PDCD4 and ABCG1) | | | | |

As shown in Table 16, we compared the predictive accuracy of the 12-gene model, the three-gene model and the two-gene model for clinical prognosis of prostate cancer patients in the BWH cohort. Remarkably, the three-gene model performed equally well with the 12-gene model (C-index 0.939, P<0.001, respectively). Although the two-gene model performed slightly less well than the 12-gene or the three-gene model (C-index=0.875, P<0.001), the difference in C-index did not reach statistical significance (P=0.134).

TABLE 16
Comparison among the prediction accuracy of the 12-gene model,
the three-gene model and the two-gene model in the BWH cohort.

| | Concor- | 95% | P-value for | P-value vs. |
|---|---|---|---|---|
| | dance | Confidence | C-index | 12-gene |
| | index | Interval | (vs. 0.5) | model |
| 12-gene model | 0.939 | 0.862-1.000 | <0.001 | |
| 3-gene model | 0.939 | 0.862-1.000 | <0.001 | N.A. |
| (PDCD4, ABCG1 | | | | |
| and KLF6) | | | | |
| 2-gene model | 0.875 | 0.713-1.000 | <0.001 | 0.134 |
| (PDCD4 and | | | | |
| ABCG1) | | | | |

N.A.: not applicable

The performances of the three-gene model and the two-gene model in the prognostic prediction of patients in the CFMC cohort were further compared. As shown in Table 17, the three-gene model performed slightly better than the two-gene model, albeit without statistically significant difference (P=0.195).

TABLE 17
Comparison among the prediction accuracy of the three-
gene model and the two-gene model in the CFMC cohort.

| | Concor- | 95% | P-value for | P-value vs. |
|---|---|---|---|---|
| | dance | Confidence | C-index | 3-gene |
| | index | Interval | (vs. 0.5) | model |
| 3-gene model | 0.951 | 0.859-1.000 | <0.0001 | |
| (PDCD4, ABCG1 | | | | |
| and KLF6) | | | | |
| 2-gene model | 0.915 | 0.801-1.000 | <0.0001 | 0.195 |
| (PDCD4 and | | | | |
| ABCG1) | | | | |

N.A.: not applicable

### Example 7

This example describes the calculation of predicted recurrence rate and expected recurrence-free survival for patients with prostate cancer based on the 12-gene prognostic model shown in Example 3.

As described in Example 3, one can measure the risk of post-operative recurrence of a given patient with prostate cancer by calculating the recurrence score based on a selected gene set (Recurrence score=Σᵢ₌₃^{k}bᵢxᵢ (Equation 1)). For a patient whose recurrence score is known, the hazard rate of recurrence at time t of said patient can be estimated by Cox regression, and the hazard rate can be expressed as h(t)=h₀(t)exp(bx), where x is the value of recurrence score, b is the regression coefficient, and h₀ (t) is the baseline hazard function. The predicted recurrence rate at time t can be estimated according to

F(t)=1−S₀(t)^{exp(bx)} (Equation 2)

Where S₀(t)=exp[−∫₀^{t}h₀(u)du] is the baseline recurrence-free function. The calculation can be carried out by commercial software such as the SPSS software (IBM) or the like. Further, the median recurrence time can be solved by F(t)=1−S₀(t)^{exp(bx)} (Equation 2) as setting F(t)=0.5.

For example, the recurrence score of a given patient in the BWH cohort can be calculated based on the transcript abundance levels of the 12 gene markers of said subject as follows:

$$x = 10.028 + ( - 1.636 ABCG 1 - 1.74 ANXA 11 + 1.811 BANF 1 - 1.345 BTD - 0.711 DSC 2 - 1.844 DUSP 2 - 1.419 IRS 1 - 1.000 KLF 4 - 2.601 KLF 6 - 2.185 PDCD 4 - 2.028 ST 6 GALNAC 2 - 1.488 ZNF 185 ) / 12 \qquad ( Equation 3 )$$

The estimated Cox regression is h(t)=h₀(t)exp(1.490x). The recurrence function can be represented by

F(t)=1−S₀(t)^{exp(1.490x)} (Equation 4)

The values of estimated S₀(t) are shown in Table 18.

TABLE 18
Baseline disease recurrence rates of patients in the BWH
cohort estimated according to the Cox regression based on
the recurrence score calculated using the 12-gene model.

| t | S₀(t) |
|---|---|
| [0, 3.32) | 1.000 |
| [3.32, 3.75) | 0.986 |
| [3.75, 6.18) | 0.966 |
| [6.18, 13.59) | 0.940 |
| [13.59, 26.45) | 0.911 |
| [26.45, 45.56) | 0.869 |
| [45.56, 55.30) | 0.811 |
| [55.30, ∞) | 0.361 |

Thus, given the transcript abundance levels of the 12 gene markers listed in

Table of a given patient, one can predict the recurrence rate and expected relapse-free survival of said patient by F(t)=1−S₀(t)^{exp(bx)} (Equation 2),

$$x = 10.028 + ( - 1.636 ABCG 1 - 1.74 ANXA 11 + 1.811 BANF 1 - 1.345 BTD - 0.711 DSC 2 - 1.844 DUSP 2 - 1.419 IRS 1 - 1.000 KLF 4 - 2.601 KLF 6 - 2.185 PDCD 4 - 2.028 ST 6 GALNAC 2 - 1.488 ZNF 185 ) / 12 \qquad ( Equation 3 )$$

and Table 12. Table 19 shows the results of prediction in four patients selected from the BWH cohort.

TABLE 19
Three-year recurrence rates and recurrence-free survival of selected
patients in the BWH cohort as predicted by the 12-gene model.

| | Patient | Patient | Patient | Patient |
|---|---|---|---|---|
| Transcript abundance level* | 1 | 2 | 3 | 4 |
| ABCG1 | 6.248 | 5.136 | 7.305 | 7.026 |
| ANXA11 | 6.858 | 9.833 | 10.391 | 9.941 |
| BANF1 | 11.440 | 12.273 | 11.489 | 11.270 |
| BTD | 10.009 | 9.802 | 10.139 | 9.870 |
| DSC2 | 7.940 | 7.779 | 7.619 | 7.677 |
| DUSP2 | 6.584 | 6.638 | 6.692 | 8.472 |
| IRS1 | 7.755 | 7.872 | 8.612 | 8.294 |
| KLF4 | 8.495 | 3.337 | 7.889 | 9.271 |
| KLF6 | 9.668 | 7.254 | 10.923 | 12.327 |
| PDCD4 | 3.970 | 9.119 | 5.989 | 6.014 |
| ST6GALNAC2 | 6.802 | 4.369 | 7.307 | 7.750 |
| ZNF185 | 6.777 | 7.883 | 5.860 | 7.894 |
| Recurrence score by the | 2.311 | 1.341 | −0.451 | −1.341 |
| 12-gene model | | | | |
| Recurrence-free survival | 0.31 | 1.13 | 3.85 | 5.55 |
| (years) | | | | |
| Predicted recurrence-free | 0.31 | 2.20 | >4.61 | >4.61 |
| survival (years) | | | | |
| Recurrence before 3 years | Yes | Yes | No | No |
| Predicted 3-year | 99% | 64% | 7% | 2% |
| recurrence rate | | | | |

*Transcript abundance levels measured by Affymetrix U95Av2 arrays (Affymetrix) and expressed as probe hybridization intensities. The data was downloaded from http://www-genome.wi.mit.edu/MPR/prostate (Singh et al., 2002).

### Example 8

This example describes the calculation of predicted recurrence rate and expected recurrence-free survival for patients with prostate cancer based on the 3-gene prognostic model as shown in Example 5.

The same principle in Example 7 can be used to apply the three-gene model, as shown in Example 5, to predict the recurrence rate and expected recurrence-free survival in patients in the CFMC cohort. According to Recurrence score=Σᵢ₌^{k}xᵢ (Equation 1), one can calculate the recurrence score of a given patient in the CFMC cohort based on the staining intensities, as represented by the H-scores, of PDCD4, ABCG1 and KLF6 in the tumor of said patient using x=7.112+(−2.771 ABCG1−2.814 KLF6−3.442 PDCD4)/3 (Equation 5).

The estimated Cox regression is h(t)=h₀(t)exp(1.235x). The recurrence function can be represented by

F(t)=1−S₀(t)^{exp(1.235x)} (Equation 6).

Table 20 shows the values of the estimated S₀(t).

TABLE 20
Baseline disease recurrence rates of patients in the CFMC
cohort estimated according to the Cox regression based on
the recurrence score calculated using the 3-gene model.

| t | S₀(t) |
|---|---|
| [0, 4) | 1.000 |
| [4, 11) | 0.991 |
| [11, 12) | 0.986 |
| [12, 16) | 0.981 |
| [16, 18) | 0.976 |
| [18, 24) | 0.970 |
| [24, 58) | 0.962 |
| [58, 60) | 0.949 |
| [60, 74) | 0.930 |
| [74, 88) | 0.889 |
| [88, ∞) | 0.694 |

Thus, for any patient in the CFMC cohort whose staining intensities of ABCG1, PDCD4 and ABCG1 are known, the predicted 3-year and 5-year recurrence rates and expected recurrence-free survival can be calculated according to x=7.112+(−2.771 ABCG1−2.814 KLF6−3.442 PDCD4)/3 (Equation 5), F(t)=1−S₀(t)^{exp(1.235x)} (Equation 6) and Table 20. Table 21 shows the results of the prediction in four patients selected from the CFMC cohort.

TABLE 21
Three-year or 5-year recurrence rates and recurrence-
free survival of selected patients in the CFMC
cohort as predicted by the 3-gene model.

| | Patient | Patient | Patient | Patient |
|---|---|---|---|---|
| H-score (per 100) | 1 | 2 | 3 | 4 |
| ABCG1 | 1.95 | 1.91 | 2.55 | 2.60 |
| PDCD4 | 1.00 | 1.75 | 2.45 | 3.00 |
| KLF6 | 1.75 | 1.10 | 2.35 | 3.60 |
| Recurrence score by the | 2.522 | 2.444 | −1.471 | −2.273 |
| 3-gene model | | | | |
| Recurrence-free survival | 1.50 | 2.00 | 5.08 | 8.50 |
| (years) | | | | |
| Predicted recurrence-free | 1.50 | 2.00 | >7.33 | >7.33 |
| survival (years) | | | | |
| Recurrence before 3 years | Yes | Yes | No | No |
| Predicted 3-year | 58.2% | 54.8% | 0.6% | 0.2% |
| recurrence rate | | | | |
| Recurrence before 5 years | Yes | Yes | No | No |
| Predicted 5-year | 80.5% | 77.4% | 1.2% | 0.4% |
| recurrence rate | | | | |

Using the same principle, one can calculate the recurrence score based on the transcript abundance levels of ABCG1, PDCD4 and KLF6 according to

x=16.682+(−1.636ABCG1−2.601KLF6−2.185PDCD4)/3 (Equation 7).

The estimated Cox regression is h(t)=h₀(t)exp(0.672x) and the recurrence function can be calculated by

F(t)=1−S₀(t)^{exp(0.672x)} (Equation 8).

Table 22 shows the values of estimated S₀(t).

TABLE 22
Baseline disease recurrence rates of patients in the BWH
cohort estimated according to the Cox regression based on
the recurrence score calculated using the 3-gene model.

| t | S₀(t) |
|---|---|
| [0, 3.32) | 1.000 |
| [3.32, 3.75) | 0.983 |
| [3.75, 6.18) | 0.962 |
| [6.18, 13.59) | 0.934 |
| [13.59, 26.45) | 0.902 |
| [26.45, 45.56) | 0.861 |
| [45.56, 55.30) | 0.815 |
| [55.30, ∞) | 0.403 |

Table 23 shows the predicted 3-year recurrence rates and recurrence-free survival in four patients selected from the BWH cohort.

TABLE 23
Three-year recurrence rates and recurrence-free survival of selected
patients in the BWH cohort as predicted by the 3-gene model.

| | Patient | Patient | Patient | Patient |
|---|---|---|---|---|
| Transcript abundance level | 1 | 2 | 3 | 4 |
| ABCG1 | 6.248 | 5.136 | 7.305 | 7.026 |
| KLF6 | 9.668 | 7.254 | 10.923 | 12.327 |
| PDCD4 | 3.970 | 9.119 | 5.989 | 6.014 |
| Recurrence score by the | 4.645 | 3.546 | −1.132 | −2.216 |
| 3-gene model | | | | |
| Recurrence-free survival | 0.31 | 1.13 | 3.85 | 5.55 |
| (years) | | | | |
| Predicted recurrence-free | 0.31 | 0.52 | >4.61 | >4.61 |
| survival (years) | | | | |
| Recurrence before 3 years | Yes | Yes | No | No |
| Predicted 3-year | 96.6% | 80.2% | 6.8% | 3.3% |
| recurrence rate | | | | |

According to the above results, the present application provides the combinations of molecular markers for predicting the clinical prognosis of prostate cancer. Compared with the known models, the present application shows improved accuracy and is suitable for clinical use.

What is claimed is:

1. A method for predicting clinical prognosis for a human subject diagnosed with prostate cancer, comprising:
detecting an expression level of a marker gene selected from a group consisting of ABCG1, PDCD4, KLF6, ST6, BTD, BANF1, IRS1, ZNF185, ANXA11, DUSP2, KLF4 and DSC2, in a biological sample containing prostate cancer cells obtained from the human subject; and
predicting a likelihood of the clinical prognosis by comparing the expression level of the marker gene with a reference level.

2. The method of claim 1, wherein the clinical prognosis is selected from the likehood of disease progression, clinical prognosis, recurrence, death or any combination thereof.

3. The method of claim 1, wherein the clinical prognosis comprises a time interval between the date of disease diagnosis or surgery and the date of disease recurrence or metastasis; a time interval between the date of disease diagnosis or surgery and the date of death of the subject; at least one of changes in number, size and volume of measurable tumor lesion of prostate cancer; or any combination thereof.

4. The method of claim 2, wherein the disease progression comprises classification of prostate cancer, determination of differentiation degree of prostate cancer cells, or a combination thereof.

5. The method of claim 1, wherein the marker gene is selected from a group consisting of ABCG1, PDCD4 and KLF6.

6. The method of claim 1, wherein the marker gene is a combination of ABCG1 and PDCD4.

7. The method of claim 1, wherein the expression level of a marker gene is determined based on a RNA transcript of the marker gene, or an expression product of the marker gene.

8. The method of claim 1, wherein the expression level of the marker gene is detected by polymerase chain reaction (PCR), northern blotting assay, RNase protection assay, microarray assay, RNA in situ hybridization, immunoblotting assay, immunohistochemistry, two-dimensional protein electrophoresis, mass spectroscopy analysis assay, or any combination thereof.

9. The method of claim 1, wherein the biological sample is obtained by aspiration, biopsy, or surgical resection.

10. The method of claim 1, wherein the reference level is determined based on the normalized expression level of the marker gene in a plurity of prostate cancer patients.

11. The method of claim 1, wherein the increased expression level of the marker gene indicates an increased or decreased likelihood of positive clinical prognosis.

12. The method of claim 11, wherein the positive clinical prognosis comprises a long-term survival without prostate cancer recurrence or a long-term overall survival of a prostate cancer patient.
